(19) United States
(12) Patent Application Publication
(10) Pub. No.: US 2015/0056194 A1
(43) Pub. Date: Feb. 26, 2015

(54) MODIFIED GREEN TEA POLYPHENOLS AND METHODS THEREOF FOR TREATING LIVER DISEASE

(71) Applicant: Georgia Regents Research Institute, Inc., Augusta, GA (US)

(72) Inventor: Stephen Hsu, Evans, GA (US)

(21) Appl. No.: 14/464,804

(22) Filed: Aug. 21, 2014

Related U.S. Application Data
(60) Provisional application No. 61/868,402, filed on Aug. 21, 2013.

(51) Int. Cl. A61K 36/82; A61K 45/06

(52) U.S. Cl. CPC A61K 36/82; A61K 45/06
USPC 424/133.1; 424/729; 424/649

(57) ABSTRACT

Methods of treating liver disease in a subject, including administering to the subject an effective amount of one or more modified green tea polyphenols to reduce, decrease, limit or prevent one or more symptoms of liver disease relative to an untreated control subject are provided. In a preferred embodiment the one or more modified green tea polyphenols are administered at a dose of 400 mg/kg body weight five times weekly. In some embodiments the disclosed methods further include administering to the subject one or more additional pharmaceutically active agents. In one embodiment the one or more additional pharmaceutically active agents is a chemotherapeutic agent.

### CROSS REFERENCE TO RELATED APPLICATIONS

This application claims the benefit of and priority to U.S. Provisional Application No. 61/868,402 filed Aug. 21, 2013.

### FIELD OF THE INVENTION

The invention relates to the field of oncology and in particular to the field of chemotherapy and chemoprevention.

### BACKGROUND OF THE INVENTION

More than 30 million people in the U.S. have liver disease. Chronic liver diseases such as cirrhosis, fatty liver disease and liver cancer result in approximately 31,000 deaths each year in the U.S.

Inflammation, steatosis and fibrosis of the liver that can occur due to long-term alcohol abuse (alcoholic fatty liver disease), or result from genetic factors, lifestyle factors such as diet, infectious diseases, as well as exposure to environmental toxins such as diethylnitrosamine (DEN) or foods contaminated with aflatoxins. Alcoholic and nonalcoholic fatty liver disease can lead to permanent liver damage as the liver enlarges and hepatocytes are replaced by non-functional scar tissue in a process known as cirrhosis (Masuoka, et al. Ann NY Acad. Sci., 1281:106-122 (2013)). Cirrhosis of the liver can lead to liver failure, liver cancer, and liver-related death. Fatty liver diseases are the leading causes of cirrhosis in the U.S.

Hepatocellular carcinoma (HCC), also known as malignant hepatoma, is the most frequent form of primary liver cancer and accounts for 70-85% of the primary malignant tumors of the liver (Perz, et al., J. Hepatol., 45:529-538 (2006)). HCC is highly prevalent in developed countries and is the sixth most common cancer and the third leading cause of cancer mortality worldwide (El-Serag, et al., Gastroenterology, 2007, 132(7): 2557-2576).

HCC is more common amongst males than females and is most prevalent between the ages of 30 and 50 (Kumar et al., Pathologic Basis of Disease (7th ed.), Saunders, pp: 914-7 (2003)). It is estimated that HCC is directly associated with 662,000 deaths worldwide per year, approximately 50% of which occur in China, where HCC is one of the deadliest cancers. Unfortunately, HCC is frequently diagnosed late because of the absence of pathognomonic symptoms and many patients have untreatable HCC when first diagnosed (Bosch, et al., Gastroenterology, 2004, 127(5 Suppl 1): S5-S16). There is currently no widespread effective chemotherapy for HCC and surgical resection is the treatment of choice for HCC in non-cirrhotic patients (Lambert, et al., Arch Biochem. Biophys., 2010, 501(1): 65-72. However, only 10-20% of hepatocellular carcinomas can be completely surgically removed and recurrence rates are as high as 50% within several years of surgery for those undergoing resection. If the cancer cannot be completely removed, the disease is usually deadly within 3 to 6 months. In view of the limited treatment and a grave prognosis of HCC, preventive controls have been emphasized and chemoprevention approaches have been considered the best strategies to protect against cancer.

Epidemiological evidence indicates the incidence and mortality of fatty liver diseases, liver cirrhosis and HCC is increasing amongst developed countries, including the United States (El-Serag, et al., Gastroenterology, 132:2557-2576 (2007)). This increase has been associated with the rising prevalence of Type 2 diabetes, which is associated with obesity (Altekruse, et al., J. Clin. Oncol., 27:1485-1491 (2009); Bosetti, et al., Hepatology, 48:137-145 (2008)), as well as infection with hepatitis B virus (HBV) and hepatitis C virus (HCV) (Jemal, et al., CA Cancer J. Clin., 2011, 61(2): 69-90). The risk of HCC in type 2 diabetics is approximately 2.5 to 7.1 greater than the risk for non-diabetics (El-Serag, et al., Clin. Gastroenterol. Hepatol., 4:369-380 (2006); Hassan, et al., Cancer (ACS) 116:1938-1946 (2010)). In the United States, an estimated 805,000-1.4 million persons are living with chronic hepatitis B infection (Weinbaum, et al., MMWR, 57:RR-8:2 (2008)), and an estimated 2.7-3.9 million persons are chronically infected with hepatitis C (Armstrong, et al., Ann. Intern. Med., 144:705-714(2006)). The known latency of HCC development from the initial HCV infection may take two to three decades (El-Serag, et al., N. Engl. J. Med. 340:745-750 (1999)).

Thus it is an object of the current invention to provide compositions and methods of use thereof to prevent and treat liver disease.

### SUMMARY OF THE INVENTION

It has been discovered that lipid soluble tea polyphenols (LTPs) prevent and reduce the symptoms of liver disease. LTPs protect the liver from oxidative stress by up-regulating the expression of antioxidative factors such as peroxiredoxin 6 (P6) and Glutathione peroxidase (GSH-Px) and increasing the total antioxidant capacity (T-AOC) of the liver tissue.

Methods of treating liver disease in a subject, including administering to the subject an effective amount of one or more modified green tea polyphenols to reduce, decrease, limit or prevent one or more symptoms of liver disease relative to an untreated control subject are provided. In a preferred embodiment the one or more modified green tea polyphenols are administered at a dose of 400 mg/kg body weight five times weekly. In some embodiments the disclosed methods further include administering to the subject one or more additional pharmaceutically active agents. In one embodiment the one or more additional pharmaceutically active agents is a chemotherapeutic agent.

The symptoms of liver diseases that can be reduced, decreased, limited or prevented by the disclosed methods include, but are not limited to increased abdominal mass, fatigue, abdominal pain, cachexia, jaundice, obstructive syndromes including lymphatic blockage and accumulation of ascites, anemia, back pain and any combination thereof. Typically the liver disease is liver cancer, fatty liver or liver cirrhosis. In a preferred embodiment the liver disease is hepatocellular carcinoma (HCC). In some embodiments the subject is asymptomatic.

Methods of prophylactically treating liver disease in a subject at risk of developing liver disease, including selecting a subject with an increased risk of developing liver disease; and administering to the subject an effective amount of one or more modified green tea polyphenols, to reduce the risk of developing liver disease relative to an untreated control are also provided. Factors associated with an increased risk of developing liver disease typically include inherited metabolic disease, liver cirrhosis, infection with Hepatitis B virus, infection with Hepatitis C virus, alcohol abuse, non-alcoholic fatty liver disease, diabetes mellitus type 2, obesity, hepatocellular adenomas, exposure to aflatoxins, exposure to environmental carcinogens, recreational drug abuse, tobacco smoking or any combination thereof. In a preferred embodiment the risk factor is infection with Hepatitis C virus.

Pharmaceutical compositions including an effective amount of one or more modified green tea polyphenols to reduce, decrease, limit or prevent one or more symptoms of liver disease in a subject relative to an untreated control subject, one or more additional pharmaceutical agents and a pharmaceutically acceptable excipient are also provided. Typically the one or more modified green tea polyphenols are administered to the subject at a daily dose equivalent to from about 0.001 to 1000 mg/kg body weight. In a preferred embodiment the one or more modified green tea polyphenols are administered to the subject at a dose equivalent to about 400 mg/kg body weight five times weekly and the one or more additional pharmaceutical agents is a chemotherapeutic agent.

### BRIEF DESCRIPTION OF THE DRAWINGS

FIG. 1 is a schematic representation of the experimental regimen. Number of weeks is shown at the top and groups are indicated on the far right. Administration throughout the thirty week period is indicated in each of groups 1-4 by shading. LTP is Lipid-soluble tea polyphenols, DEN is diethylnitrosamine and PB is Phenobarbital.

FIGS. 2A-2D are four micrograph images of representative histologic liver tissue slices from animals in group 1; normal control (FIG. 2A), group 2; 0 mg/kg LTP (FIG. 2B), group 3; 40 mg/kg LTP (FIG. 2C) and group 4; 400 mg/kg LTP (FIG. 2D), respectively. Tissues are stained with Hematoxylin and Eosin (HE), and images are taken at 400× magnification.

FIGS. 3A-3D are four micrograph images of representative histologic liver tissue slices from animals in group 1; normal control (FIG. 3A), group 2; 0 mg/kg LTP (FIG. 3B), group 3; 40 mg/kg LTP (FIG. 3C) and group 4; 400 mg/kg LTP (FIG. 3D), respectively. Tissues are stained with Masson's trichrome stain, and images are taken at 200× magnification.

FIGS. 4A-4D are four micrograph images of representative histologic liver tissue slices immune-stained for Glutathione-S Transferase protein (GST-P; dark regions). Tissues are taken from animals in group 1; normal control (FIG. 4A), group 2; 0 mg/kg LTP (FIG. 4B), group 3; 40 mg/kg LTP (FIG. 4C) and group 4; 400 mg/kg LTP (FIG. 4D), respectively. Images are taken at 100× magnification. FIG. 4E is histogram showing the area of GST-P-positive foci on histologic liver tissue slices from groups 0 mg/kg LTP of FIG. 4B (group 2); 40 mg/kg LTP of FIG. 4C (group 3) and 400 mg/kg LTP of FIG. 4D (group 4). FIG. 4F is a histogram showing the quantitation of GST-P-positive foci for representative histologic liver tissue slices from groups 0 mg/kg LTP of FIG. 4B (group 2); 40 mg/kg LTP of FIG. 4C (group 3) and 400 mg/kg LTP of FIG. 4D (group 4. Significance was determined by one-way analysis of variance. Data are mean±SE.* p<0.05vs LTPs for 0 mg/kg group.

FIGS. 5A-5D are four micrograph images of representative histologic liver tissue slices immune-stained for proliferating cell nuclear antigen (PCNA) (foci indicated with arrows). Tissues are taken from animals in group 1; normal control (FIG. 5A), group 2; 0 mg/kg LTP (FIG. 5B), group 3; 40 mg/kg LTP (FIG. 5C) and group 4; 400 mg/kg LTP (FIG. 5D), respectively. Images are taken at 400× magnification.

FIG. 6 is a histogram showing the quantitation of PCNA positive foci on histologic liver tissue slices from each group. Data are mean±SE. * p<0.05vs Normal control group, #p<0.05 vs DEN/PB group.

FIGS. 7A-7D are four micrograph images of representative histologic liver tissue slices immune-stained for 8-hydroxy-2′-deoxyguanosine (8-OHdG) (dark regions). Tissues are taken from animals in group 1; normal control (FIG. 7A), group 2; 0 mg/kg LTP (FIG. 7B), group 3; 40 mg/kg LTP (FIG. 7C) and group 4; 400 mg/kg LTP (FIG. 7D), respectively. Images are taken at 400× magnification.

FIG. 8 is a histogram showing the relative expression of 8-OHdG in histologic liver tissue slices from each group. Data are mean±SE. # p<0.05 vs Normal control group, *p<0.05 vs DEN/PB group.

FIG. 9 is a line graph of animal growth (body weight) over the entire time of the study (weeks). The effects LTP on body weight gain during DEN-induced hepatocarcinogenesis in rats from group 1; normal control ( [symbol] ), group 2; 0 mg/kg ( [symbol] ), group 3; 40 mg/kg ( [symbol] ) and group 4; 400 mg/kg ( [symbol] ) are indicated, respectively. Statistical significance was determined by general linear model/repeated measures. *, p<0.05 compared to 0 mg/kg group.

FIGS. 10A-10B are histograms showing the absolute (FIG. 10A) and relative (FIG. 10B) weight of liver tissues from rats in each group in response to GTP and LTP during DEN-induced hepatocarcinogenesis. Statistical significance was determined by ANOVA. *, p<0.05 compared to GTP 0 mg/kg group; #, p<0.05 compared to LTP 0 mg/kg group. Data are the Mean±SE.

FIGS. 11A-11B are panels of micrograph images of representative histologic liver tissue slices from animals at the end of the study. Animals received 0 mg/kg, 40 mg/kg and 400 mg/kg of GTP (upper panel) or LTP (lower panel), respectively, stained with Hematoxylin and Eosin (HE) and taken at 400× magnification (FIG. 11A), or from an Electron Microscope at 8900× magnification (FIG. 11B).

FIGS. 12A-12B are histograms showing the total antioxidant capacity (T-AOC, standardized to model group) (FIG. 12A) and the activity of Glutathione peroxidase (GSH-Px activity, standardized to model group) (FIG. 12B), respectively, in the liver of rats following exposure to GTP/LTP during DEN-induced hepatocarcinogenesis. *, p<0.05 compared to GTP 0 mg/kg group; #, p<0.05 compared to LTP 0 mg/kg group. Data are shown as the Mean±SE. Statistical significance was determined by ANOVA.

FIGS. 13A-13B are panels of micrograph images of representative histologic liver tissue slices from animals at the end of the study. Animals received 0 mg/kg, 40 mg/kg and 400 mg/kg of GTP (upper panel) or LTP (lower panel), respectively, immune stained for Nrf2 (FIG. 13A), or for P6 (FIG. 13B). Images are taken at 400× magnification. FIG. 13C is a histogram showing the percentage of hepatic Nrf2 positive cells (per 1000 cells) by dose of GTP or LTP, respectively. Data are mean±S.E. (n=4, *, p <0.05 compared to GTP 0 mg/kg group). FIG. 13D is a histogram showing the percentage of hepatic P6 positive cells (per 1000 cells) by dose of GTP or LTP, respectively. Data are mean±S.E. (n=4, #, p <0.05 compared to LTP 0 mg/kg group).

### DETAILED DESCRIPTION OF THE INVENTION

### I. Definitions

The term “LTP” refers to lipid soluble green tea polyphenols, such as in Formula 1.

The term “effective amount” or “therapeutically effective amount” means a dosage sufficient to provide treatment of the disease state being treated or to otherwise provide a desired pharmacologic and/or physiologic effect. The precise dosage will vary according to a variety of factors such as subject-dependent variables (e.g., age, immune system health, etc.), the disease, and the treatment being effected.

The terms “individual”, “subject” and “patient” are used interchangeably herein, and refer to a mammal, including, but not limited to, humans, rodents, such as mice and rats, and other laboratory animals.

The terms “treat”, “treatment” and “treating” refer to the reduction or amelioration of the progression, severity and/or duration of a disease or disorder, delay of the onset of a disease or disorder, or the amelioration of one or more symptoms (preferably, one or more discernible symptoms) of a disease or disorder, resulting from the administration of one or more therapies (e.g., one or more therapeutic agents such as a compound of the invention). The terms “treat”, “treatment” and “treating” also encompass the reduction of the risk of developing a disease or disorder, and the delay or inhibition of the recurrence of a disease or disorder.

The terms “enhance”, “increase”, “stimulate”, “promote”, “decrease”, “inhibit” or “reduce” are used relative to a control. Controls are known in the art. For example an increase response in a subject or cell treated with a compound is compared to a response in subject or cell that is not treated with the compound.

The term “pharmaceutically acceptable carrier” or “pharmaceutically acceptable excipient” means one or more carrier ingredients approved by a regulatory agency of the Federal or a state government or listed in the U.S. Pharmacopoeia or other generally recognized pharmacopoeia for use in animals, mammals, and more particularly in humans. Non-limiting examples of pharmaceutically acceptable carriers include liquids, such as water and oils, including those of petroleum, animal, vegetable, or synthetic origin. Water is preferred vehicle when the compound of the invention is administered intravenously. Saline solutions and aqueous dextrose and glycerol solutions can also be employed as liquid vehicles, particularly for injectable solutions.

The term “in combination” refers to the use of more than one therapeutic agent. The use of the term “in combination” does not restrict the order in which said therapeutic agents are administered to a subject.

“Localization Signal” or “Sequence or Domain or Ligand” or “Targeting Signal or Sequence or Domain or Ligand” are used interchangeably and refer to a signal that directs a molecule to a specific cell, tissue, organelle, or intracellular region. The signal can be polynucleotide, polypeptide, or carbohydrate moiety or can be an organic or inorganic compound sufficient to direct an attached molecule to a desired location.

### II. Methods for Treating Liver Disease

It has been discovered that modified lipid-soluble green tea polyphenols (LTPs) dramatically reduce the symptoms of liver diseases such as liver cancer, fatty liver and liver cirrhosis. Methods of treating liver disease in a subject, including administering to the subject an effective amount of one or more modified green tea polyphenols to reduce, decrease, limit or prevent one or more symptoms of liver disease relative to an untreated control subject are provided.

LTPs counteract the effects of oxidative stress that are associated with the initiation and development of liver diseases such as liver cancer, fatty liver and liver cirrhosis. The antioxidant effects of LTPs were significantly greater than those of naturally occurring green tea polyphenols (GTPs). LTPs inhibited hepatic damage in the livers of rats treated with diethylnitrosamine and Phenobarbital (DEN/PB). This inhibition is associated with reduced cell proliferation, decreased fibrosis and down regulation of DNA oxidative damage markers.

A. Methods of Using LTPs

Methods of using LTPs to treat and prevent liver disease are provided. The methods typically include treating liver disease in a subject, including administering to the subject an effective amount of one or more modified green tea polyphenols to reduce, decrease, limit or prevent one or more symptoms of liver disease relative to an untreated control subject. The liver disease, disorder or condition is typically a consequence of oxidative stress. The liver disease, disorder or condition may result from oxidative stress as a secondary manifestation of a symptom such as inflammation. The liver disease, disorder or condition may itself cause oxidative stress and may result in the initiation, development or advancement of liver cancer.

The methods of use disclosed herein typically include treating a subject with an effective amount of one or more LTPs, or a derivative, analog or prodrug, or a pharmacologically active salt thereof to prevent, reduce or decrease the symptoms of liver disease. In preferred embodiments, one or more LTPs, or a derivative, analog or prodrug, or a pharmacologically active salt thereof is administered to a subject in need thereof. The subject can have a liver disease, disorder or condition caused or exacerbated by oxidative stress.

1. Effective Amounts

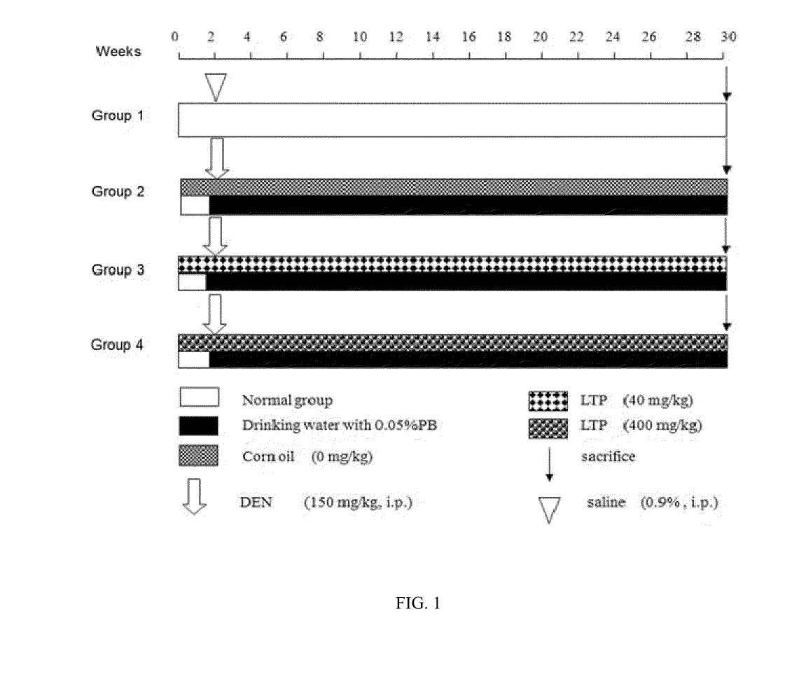

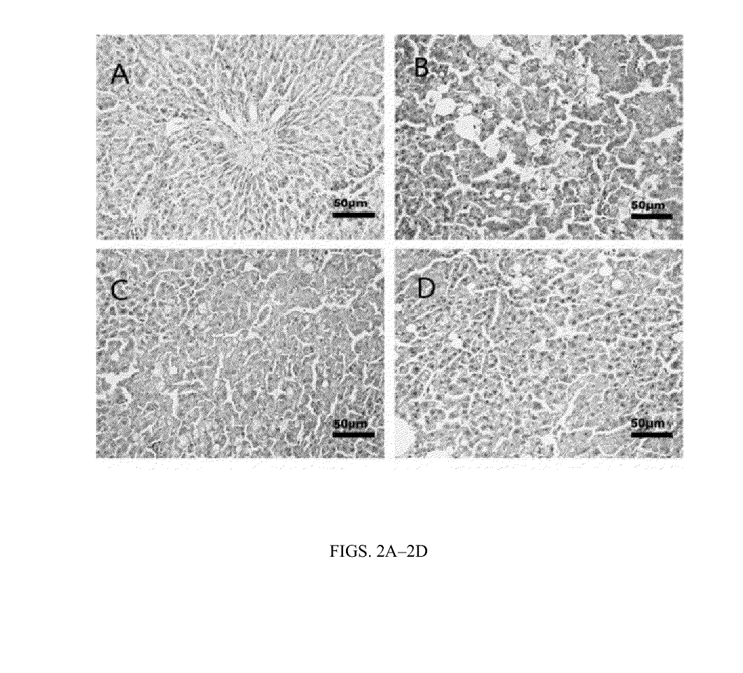

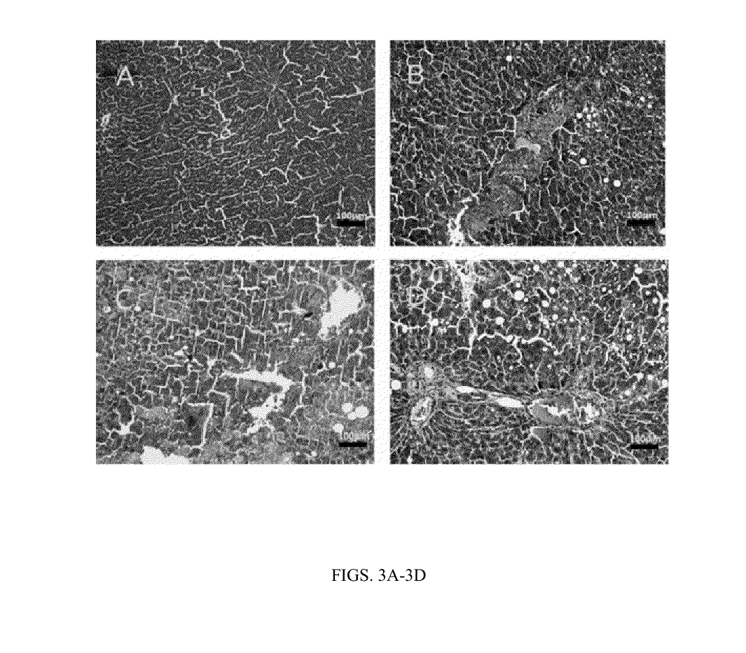

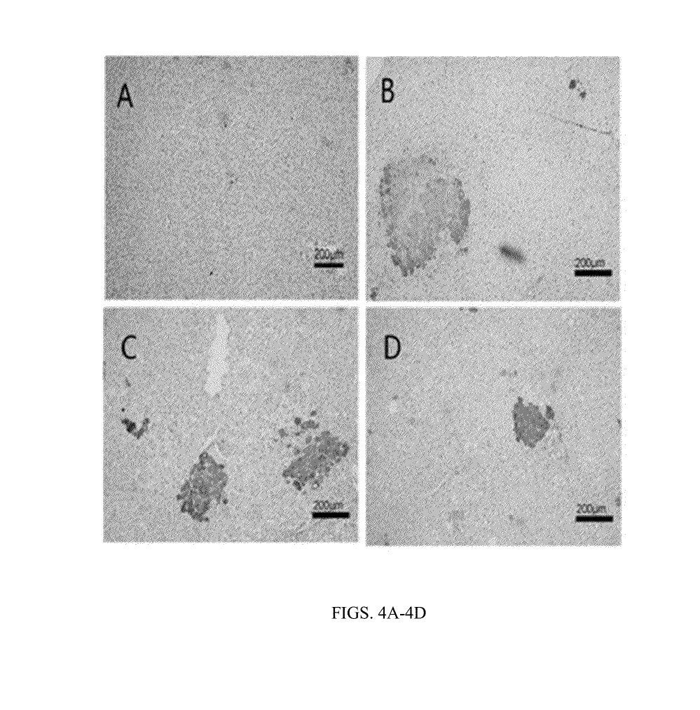

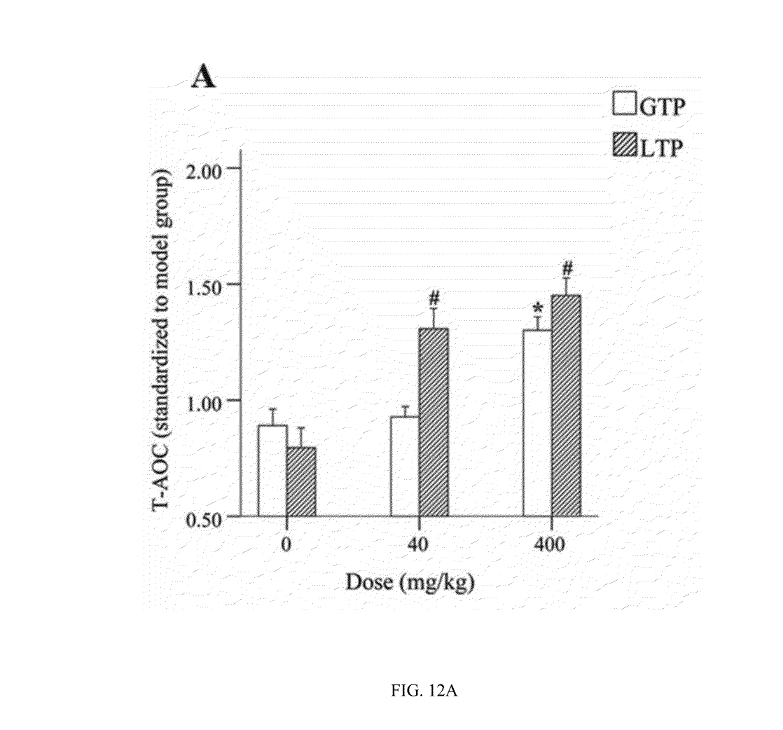

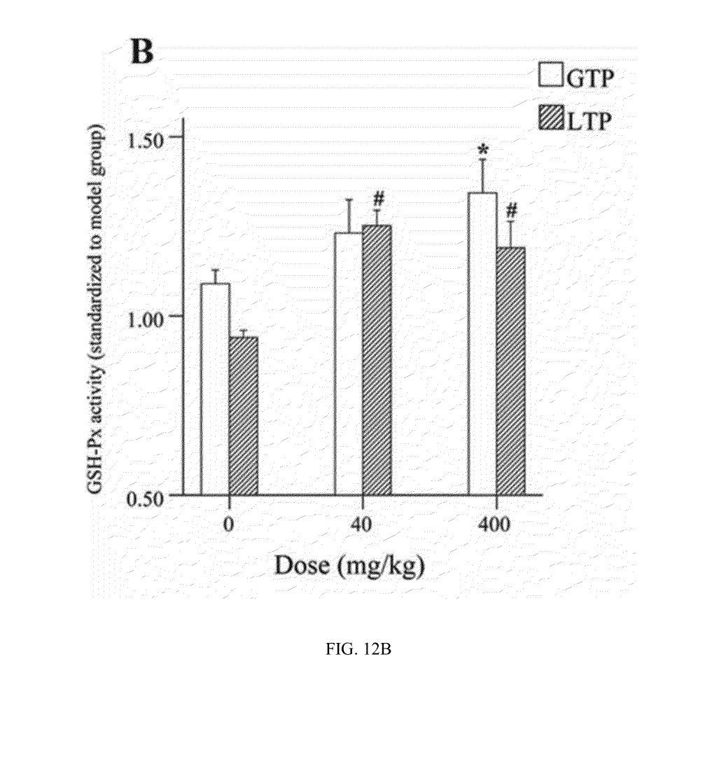

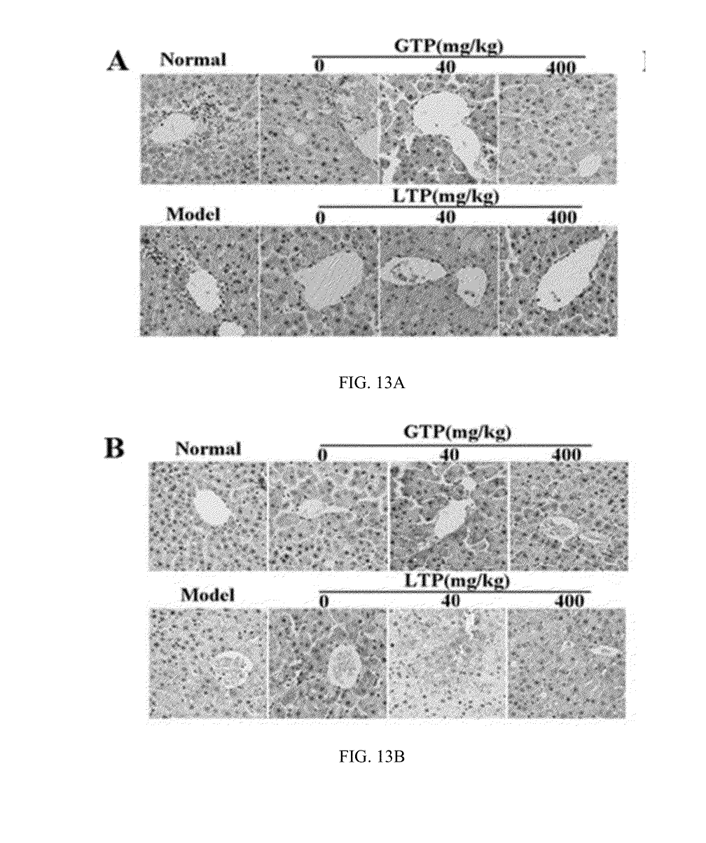

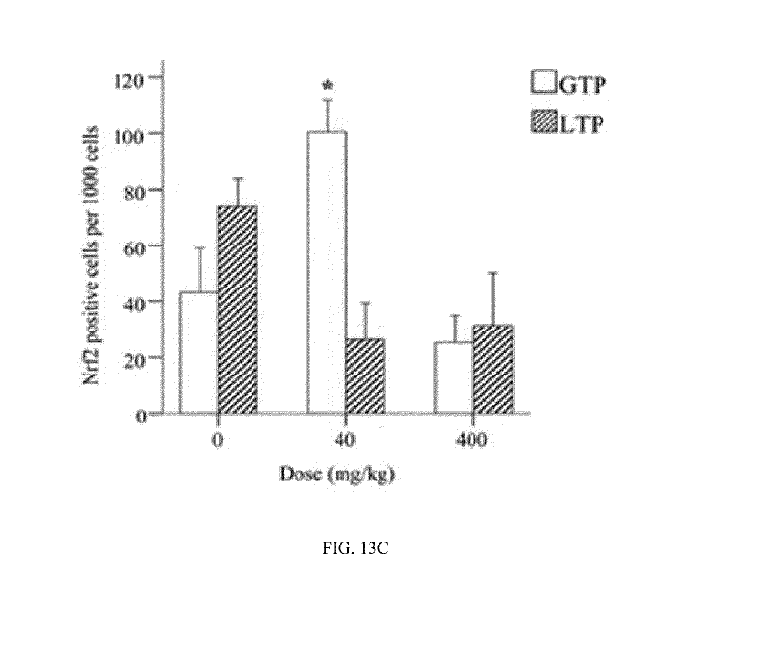

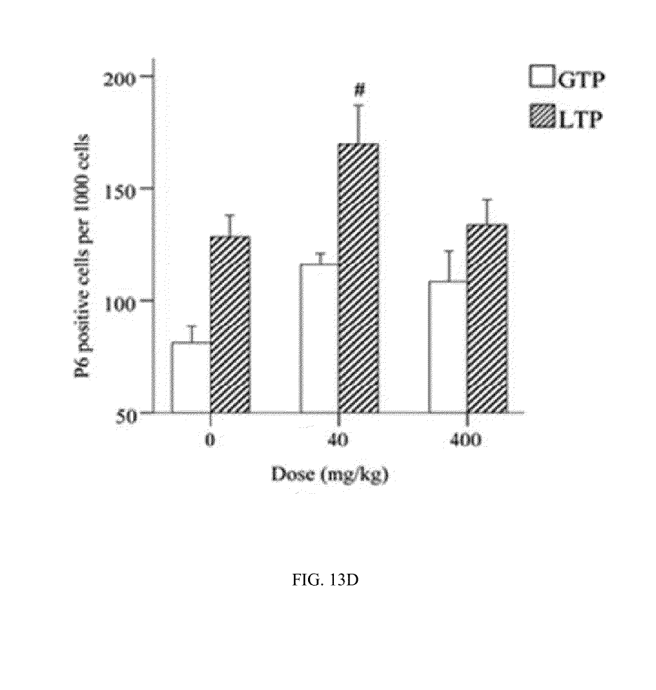

As used herein the term “effective amount” or “therapeutically effective amount” means a dosage sufficient to treat, inhibit, or alleviate one or more symptoms of the disorder being treated or to otherwise provide a desired pharmacologic and/or physiologic effect. The amount of one or more LTPs administered to a subject is typically enough to prevent, reduce, decrease, or inhibit the symptoms of liver disease. The Examples below illustrate that one or more LTPs inhibited the progression of liver cancer in a concentration-dependent manner (FIGS. 1 and 2) and caused an increase in the expression of the oxidative stress response proteins Preoxiredoxin 6 (P6) and Glutathione Peroxidase (GSH-Px) (FIGS. 12B and 13B). Therefore, in some embodiments one or more LTPs, or a derivative, analog or prodrug or salt thereof can reduce or inhibit oxidative stress; increase the expression of oxidative stress response elements such as P6 and GSH-Px; reduce or inhibit free radicals, such as hydroxyl radicals, or a combination thereof. In some embodiments, one or more LTPs, or a derivative, analog or prodrug thereof improves hepatocellular structure, reduces steatosis of hepatocytes, reduces fibrosis, or a combination thereof in a subject with liver disease relative to an untreated control subject.

In preferred embodiments, one or more LTPs, or a derivative, analog or prodrug, increases the total anti-oxidative capacity (TOAC) in a subject. As illustrated in the Examples below, in contrast to the control group, LTPs had a positive correlation with the TOAC on rat cells following DEN/PB induced HCC (FIG. 12A). As discussed above, over-expression of the oxidative stress response elements P6 and GSH-Px is associated with a reduction in tumor development and can, therefore assist in the prevention and treatment of liver cancer. Therefore, in some embodiments, one or more LTPs, or a derivative, analog or prodrug thereof can prevent, reduce or otherwise decrease the symptoms of liver cancer.

2. Controls

The effect of one or more LTPs can be compared to a control. For example, in some embodiments, one or more of the pharmacological or physiological markers or pathways affected by treatment with one or more LTPs is compared to the same pharmacological or physiological marker or pathway in untreated control subjects. In preferred embodiments the subject suffers the same disease or conditions as the treated subject. For example, subjects treated with one or more LTPs can be compared to subjects treated with pharmaceutical agents known to prevent, reduce or decrease the symptoms of liver disease. The cells or subjects treated with other agents known to prevent, reduce or decrease the symptoms of liver disease can have a greater increase in oxidative stress, or a greater increase in tumorigenic markers than do cells or subjects treated with one or more LTPs, or a derivative, analog or prodrug thereof.

In preferred embodiments, one or more LTPs, or a derivative, analog or prodrug thereof is effective to reduce, inhibit, or delay one or more symptoms of a liver disease, disorder or condition in a subject. Liver diseases, disorders or conditions that can be prevented, reduced or treated using the disclosed compositions are discussed in more detail below.

One or more LTPs, or a derivative, analog or prodrug, or a pharmacologically active salt thereof can be administered enterally or parenterally. One or more LTPs, or a derivative, analog or prodrug, or a pharmacologically active salt thereof can be part of a pharmaceutical composition that includes a pharmaceutically acceptable carrier.

3. Therapeutic Administration Pharmaceutical compositions including one or more LTPs, or a derivative, analog or prodrug, or a pharmacologically active salt thereof, may be administered in a number of ways depending on whether local or systemic treatment is desired, and depending on the area to be treated. For all of the disclosed compounds, as further studies are conducted, information will emerge regarding appropriate dosage levels for treatment of various conditions in various patients, and the ordinary skilled worker, considering the therapeutic context, age, and general health of the recipient, will be able to ascertain proper dosing. The selected dosage depends upon the desired therapeutic effect, on the route of administration, and on the duration of the treatment desired. Generally dosage levels of 0.001 to 100 mg/kg of body weight daily are administered to mammals. Generally, for intravenous injection or infusion, dosage may be lower. Preferably, the compositions are formulated to achieve a one or more LTPs serum level of about 1 to about 1000 μM.

In some embodiments the disclosed methods and compositions are administered to a subject for therapeutic treatment of a liver condition, disease or disorder. Typically the subject has been diagnosed with a liver disease. Therapeutic treatment can occur at any time following initiation of a liver disease. In one embodiment therapeutic treatment may occur following initiation of symptoms of liver disease. In another embodiment, therapeutic administration may occur following identification of signs and markers for disease in the absence of symptoms.

In some embodiments the disclosed methods and compositions are administered to a subject for prophylactic treatment of a condition, disease or disorder. Methods for the treatment of a disease, disorder or condition in a subject wherein the subject has not been diagnosed with a disease or who does not have symptoms of a disease are provided. In a preferred embodiment the subject has one or more risk factors associated with the development of liver disease.

B. Diseases to be Treated

The methods and compositions disclosed herein can be used to treat or prevent a variety of diseases and disorders in which an increase in total anti-oxidative capacity (TAOC) is desirable. The compositions and methods disclosed herein can be used to treat any disease or disorder in which increased oxidative stress plays a pathogenic role in the disease or disorder.

1. Liver Disease

The disclosed methods and compositions can be used to reduce, decrease, prevent or otherwise limit the initiation, development, progression, signs or symptoms of diseases and disorders of the liver. TOAC is important in preventing oxidative stress in diseases of the liver. Accordingly, if liver diseases or disorders result from, or are exacerbated by an increase in oxidative stress, it is desirable to promote the local production of oxidative stress response mechanisms by LTPs in the liver. The oxidative stress can result from inflammation of the liver or from inflammation of tissues proximal to the liver and the methods disclosed herein can be used to treat liver diseases or disorders in which inflammation and increased oxidative stress plays a pathogenic role in the diseases or disorder. In some embodiments inflammation of the liver is caused by the presence of an infectious agent. The infectious agent is typically a virus, bacterium, fungus, protozoan, or parasite. In other embodiments inflammation of the liver is caused by a toxin. The toxin is typically alcohol, diethylnitrosamine (DEN) or an aflatoxin.

LTPs can be administered locally or systemically in an effective amount to increase the local or systemic oxidative stress response elements, for example, in order to treat or prevent diseases of the liver. In a preferred embodiment the one or more modified green tea polyphenols are administered at a dose of 400 mg/kg body weight five times weekly. Exemplary liver diseases and disorders that can be treated by LTPs are provided below.

a) Liver Cancer

The disclosed methods and compositions can be used to reduce, decrease, prevent or otherwise limit the initiation, development, progression, signs or symptoms of liver cancers. In some embodiments, the liver cancers to be treated are asymptomatic liver cancers. In some embodiments, the liver cancer has been identified by detection of diagnostic markers associated with the initiation, development or progression of liver cancers. In further embodiments risk factors for the development of liver cancers are used as a mechanism to identify subjects that can benefit from prophylactic treatment with the disclosed methods and compositions.

b. Liver Cirrhosis

The disclosed methods and compositions can be used to reduce, decrease, prevent or otherwise limit the initiation, development, progression, signs or symptoms of liver cirrhosis.

Liver cirrhosis is a disease in which liver cells become damaged and are replaced by scar tissue. Factors that contribute to cirrhosis include but are not limited to infectious diseases, alcohol abuse, recreational drug abuse and non-fatty liver diseases. Liver cirrhosis is associated with the development of liver cancer. Chronic infection with hepatitis C virus (HCV) has been recognized as an increased risk of HCC. Approximately 20% of HCV-infected individuals have diseases that progress to cirrhosis, and about 40% of these patients develop HCC after a mean of 10-15 years. Cirrhosis of the liver can also be caused by infection with Hepatitis B virus (HBV).

c. Fatty Liver Disease

The disclosed methods and compositions can be used to reduce, decrease, prevent or otherwise limit the initiation, development, progression, signs or symptoms of fatty liver disease. The fatty liver disease may be a result of alcohol abuse, which is a leading cause of cirrhosis in the United States.

The fatty liver disease may also be non-alcoholic fatty liver disease. Cirrhosis of the liver can be caused by non-alcoholic fatty liver disease, which is a condition in which people who consume little or no alcohol develop a fatty liver. Non-alcoholic fatty liver disease is common in obese people. People with a type of this disease known as non-alcoholic steatohepatitis (NASH) might go on to develop cirrhosis. Type 2 diabetes has been linked with an increased risk of liver cancer, especially in patients who also have other risk factors such as heavy alcohol use and/or chronic viral hepatitis. This risk may be increased because people with type 2 diabetes tend to be overweight or obese, which in turn can cause liver problems.

In some embodiments LTPs, or derivatives, analogs or prodrugs, or pharmacologically active salts thereof are used prophylactically. Accordingly, LTPs can be administered daily in the absence of the symptoms or markers of liver disease, to promote general liver health and to prevent the development of liver disease. 2. Other Diseases Associated with Oxidative Stress

Other diseases and disorders that can be treated by the disclosed methods and compositions include diseases that are known to be caused by or otherwise associated with oxidative stress.

a) Other Cancers

In some embodiments the diseases and disorders that can be treated by the disclosed methods and compositions are other forms of cancer. Other forms of cancer that are associated with oxidative stress include but are not limited to cancer of the stomach, prostate, breast, lung, bladder colorectal cancer, uterine cancer, ovarian cancer, lymphoma and skin cancer.

C. Markers for Liver Disease

In some embodiments, risk factors for the development of liver diseases are used as a mechanism to identify subjects that can benefit from prophylactic treatment with the disclosed methods and compositions. Liver disease can be identified by detection of diagnostic markers associated with the initiation, development or progression of liver disease, as described below. 1. Indications and Diagnostic Markers of Liver Diseases

The signs or indications of liver diseases can include, but are not limited to local chronic inflammation of liver tissues, fibrosis of the liver tissue, hepatomegaly, immune evasion by liver cells, deregulated metabolism of liver cells, hepatocyte steatosis and loss of normal hepatocyte architecture, sustained angiogenic ability of liver cells, self-sufficiency of growth signals in liver tissues, insensitivity of liver cells to anti-growth signals, evasion of apoptosis by liver cells, limitless reproductive potential of liver cells, capability of liver cells to invade other tissues and metastasize and chromosomal abnormalities amongst liver cells.

Patients with liver cancers, such HCC are usually asymptomatic during the early stages of disease. Thus, 80% of patients with HCC will be diagnosed with advanced stage disease. Clinical investigation of the signs and indications of liver cancers as well as the establishment of diagnostic markers for the early identification of liver cancers can be used to identify subjects that can benefit from the disclosed methods and compositions.

Diagnostic markers for liver diseases can include, but are not limited to the expression of CK-7, CK-19 and CD34 molecules at the surface of hepatocyte progenitor cells (Durnez, et al., Hepatology, 49:138-151 (2006)), serum alpha-fetoprotein (AFP), Lectin-bound alpha-fetoprotein (AFP-L3) and Des-gamma carboxyprothrombin (DCP), surveillance with ultrasonography, carbohydrate-lectin based analytical markers and serum antibodies for disialosyl galactosyl globoside (DSGG), and fucosyl mono-sialo-tetra-hexosyl-ganglioside (fucosyl-GM1) (Wu, et al., PLoS ONE, 7:e39466 (2012)).

2. Risk Factors for Liver Diseases

The disclosed methods and compositions can be used prophylactically to prevent or reduce the incidence of the development of liver disease, including liver cancers. Subjects at risk of developing liver diseases and liver cancers can benefit from prophylactic treatment with the disclosed methods and compositions. In some embodiments the disclosed methods and compositions prevent or reduce the development of liver disease in subjects with factors associated with development of liver disease compared with untreated control subjects.

a. Liver Inflammation

In some embodiments the risk factor for liver disease is liver inflammation. People with liver inflammation have increased risk of liver cirrhosis and liver cancer. Most but not all people who develop liver cancer already have evidence of cirrhosis and it has been estimated that liver cirrhosis is present in approximately 90% of HCC cases (Okuda, et al., Hepatology, 15:948-63 (1992)).

b. Hepatitis Viruses

One risk factor for the development of liver cirrhosis and liver cancer is infection with Hepatitis virus. Chronic infection with hepatitis C virus (HCV) has been recognized as an increased risk of HCC. Approximately 20% of HCV-infected individuals have diseases that progress to cirrhosis, and about 40% of these patients develop HCC after a mean of 10-15 years. Cirrhosis of the liver can also be caused by infection with Hepatitis B virus (HBV).

c. Genetic factors

In some embodiments the risk factor for liver disease is a genetic characteristic of the subject. Genetic factors associated with liver cirrhosis and liver cancer can be an inherited metabolic disease. Inherited metabolic disease that are associated with liver cirrhosis and liver cancers include, but are not limited to hemochromatosis, glycogen storage disease, Tyrosinemia, Alpha1-antitrypsin deficiency, Porphyria cutanea tarda, acute and chronic hepatic porphyrias (acute intermittent porphyria, porphyria cutanea tarda, hereditary coproporphyria, variegate porphyria), Gilbert's syndrome, hemochromatosis, Wilson disease and tyrosinemia type I. Both active and latent genetic carriers of acute hepatic porphyrias are at increased risk for hepatocellular carcinoma, although latent genetic carriers have developed the cancer at a later age than those with classic symptoms. Patients with acute hepatic porphyrias should be monitored for hepatocellular carcinoma.

d. Diabetes Mellitus Type 2

In one embodiment the risk factor for liver disease is diabetes mellitus type 2. Type 2 diabetes has been linked with an increased risk of liver cancer, usually in patients who also have other risk factors such as heavy alcohol use and/or chronic viral hepatitis. This risk may be increased because people with type 2 diabetes tend to be overweight or obese, which in turn can cause liver problems.

e. Other Risk Factors for Liver Disease

In a further embodiment the risk factor for HCC is one or more factors taken from the list including obesity, hepatocellular adenoma, tobacco smoking, exposure to environmental toxins such as diethylnitrosamine (DEN), consumption of food containing carcinogenic substances such as aflatoxins as well as other diseases and conditions of the liver. In one embodiment the risk factor for HCC is a disease or conditions of the liver including but not limited to biliary artesia, infantile cholestasis, Budd-Chiari syndrome, primary sclerosing cholangitis and autoimmune diseases such as autoimmune hepatitis.

LTPs can be used to increase oxidative response elements either locally or systemically in order to prevent, reduce, limit or delay the symptoms of liver diseases or disorders. Typical symptoms of liver diseases include, but are not limited to increased abdominal mass, fatigue, abdominal pain, cachexia, jaundice, obstructive syndromes including lymphatic blockage and accumulation of ascites, anemia and back pain (Sun, et al., Clin J. Oncol. Nurs., 12:759-766 (2008)).

D. Combination Therapies

The compositions of LTPs disclosed herein can be used in combination with one or more additional therapeutic agents. The term “combination” or “combined” is used to refer to either concomitant, simultaneous, or sequential administration of two or more agents. Therefore, the combinations can be administered either concomitantly (e.g., as an admixture), separately but simultaneously (e.g., via separate intravenous lines into the same subject), or sequentially (e.g., one of the compounds or agents is given first followed by the second). The additional therapeutic agents can be administered locally or systemically to the subject, or coated or incorporated onto, or into a device.

The additional agent or agents can be a second therapeutic that is used to enhance the therapeutic effect of LTPs, or a derivative, analog or prodrug, or a pharmacologically active salt thereof by targeting a second molecular pathway relevant to the disease, disorder, or condition being treated. In some embodiments, the one or more additional agent is a conventional therapeutic agent for the disease, disorder, or condition to be treated. For example, if the disease to be treated is cancer, a conventional therapeutic agent can be chemotherapy.

It is believed that LTPs can be used to increase the total antioxidant capacity of the body. Therefore, in some embodiments, the second (conventional) therapeutic agent is used at a lower dosage or for a shorter duration than if it used alone. For example, if LTP is administered in combination with a chemotherapeutic agent to target cancer cells, the chemotherapeutic agent can be used at lower dosage or for a shorter duration than if the chemotherapeutic agent is administered without LTPs or a derivative, analog or prodrug, or a pharmacologically active salt thereof. 1. Chemotherapeutic Agents

Additional therapeutic agents can also include conventional cancer therapeutics such as chemotherapeutic agents, cytokines, chemokines, and radiation therapy. The majority of chemotherapeutic drugs can be divided in to: alkylating agents, antimetabolites, anthracyclines, plant alkaloids, topoisomerase inhibitors, and other antitumor agents. All of these drugs affect cell division or DNA synthesis and function in some way. Additional therapeutics include monoclonal antibodies and the new tyrosine kinase inhibitors e.g., imatinib mesylate (GLEEVEC® or GLIVEC®), which directly targets a molecular abnormality in certain types of cancer (chronic myelogenous leukemia, gastrointestinal stromal tumors).

In a preferred embodiment the additional therapeutic agent is a chemotherapeutic agent. Representative chemotherapeutic agents include, but are not limited to sorafenib, erlotinib hydrochloride, cisplatin, cetuximab, sunitinib, bevacizumab, carboplatin, oxaliplatin, mechlorethamine, cyclophosphamide, chlorambucil, vincristine, vinblastine, vinorelbine, vindesine, taxol and derivatives thereof, irinotecan, topotecan, amsacrine, etoposide, etoposide phosphate, teniposide, epipodophyllotoxins, trastuzumab, rituximab and combinations thereof.

2. Drugs to Treat Infection

a) Drugs to Treat Viral Infection

In some embodiments the additional therapeutic agents are agents that treat viral infection. Exemplary antiviral drugs include Acyclovir, Adefovir, Amantadine, Amprenavir, Ampligen, Arbidol, Atazanavir, Atripla, Balavir, Boceprevirertet, Cidofovir, Combivir, Darunavir, Delavirdine, Didanosine, Docosanol, Edoxudine, Efavirenz, Emtricitabine, Enfuvirtide, Entecavir, Famciclovir, Fomivirsen, Fosamprenavir, Foscarnet, Fosfonet, Ganciclovir, Ibacitabine, Imunovir, Idoxuridine, Imiquimod, Indinavir, Inosine, Lamivudine, Lopinavir, Loviride, Maraviroc, Moroxydine, Methisazone, Nelfinavir, Nevirapine, Nexavir, Oseltamivir, Peginterferon alfa-2a, Penciclovir, Peramivir, Pleconaril, Podophyllotoxin, Raltegravir, Ribavirin, Rimantadine, Ritonavir, Pyramidine, Saquinavir, Stavudine, Tea tree oil, Telaprevir, Tenofovir, Tenofovir disoproxil, Tipranavir, Trifluridine, Trizivir, Tromantadine, Truvada, Valaciclovir, Valganciclovir, Vicriviroc, Vidarabine, Viramidine, Zalcitabine, Zanamivir and Zidovudine.

b) Drugs to Treat Bacterial Infection

In some embodiments the additional therapeutic agents are agents that treat bacterial infection, such as antibiotics. Exemplary antibiotics include members of the groups of Tetracyclines, Sulfonamides, Quinolones, Penicillin combinations, Penicillins, Oxazolidonones, Nitrofurans, Monobactams, Macrolides, Lincosamides, Cephalosporins, Carbapenems, Ansamycins and Aminoglycosides.

c) Drugs to Treat Fungal Infection

In some embodiments the additional therapeutic agents are agents that treat fungal infection. Exemplary antifungal drugs include Azole drugs, which inhibit ergosterol biosynthesis and are the most widely deployed antifungals in the clinic, and echinocandins, which inhibit beta(1, 3)-glucan synthesis and are the only modern class of antifungals to reach the clinic in decades. (Cowen et al, Proc. Natl. Acad. Sci. USA, 106:2813-23 (2009)).

Representative antifungal drugs include, but are not limited to Clotrimazole, Posaconazole, Ravuconazole, Econazole, Ketoconazole, Voriconazole, Fluconazole, Itraconazole, Tebuconazole and Propiconazole. In another embodiment the additional therapeutic agent is an echinocandin. Representative echinocandins include, but are not limited to pneumocandins, Echinocandin B, Cilofungin, Caspofungin, Micafungin (FK463) and Anidulafungin (VER-002, V-echinocandin, LY303366).

3. Other Active Agents

Other active agents that can be used alone, or in combination with LTPs include, but are not limited to, vitamin supplements, appetite-stimulating medications, medications that help food move through the intestine, nutritional supplements, anti-anxiety medication, anti-depression medication, anti- coagulants, clotting factors, antiemetic medications, antidiarrheal medications, anti-inflammatories, drugs that suppress the immune system, steroids such as corticosteroids or drugs that mimic progesterone, omega-3 fatty acids supplements, eicosapentaenoic acid supplements, anti-inflammatories, anabolic agents, psycho-stimulants, selective androgen-receptor modulators, anti-depressant medications, anti-anxiety medications and analgesics.

### III. Compositions for Treating Liver Disease

It has been discovered that LTPs protect the liver from damage caused by exposure to toxins and can reverse the effects of toxin-induced liver disease. LTPs decreased fibrosis and resulted in the down-regulation of DNA oxidative damage markers in a rat model for the development of liver cancer. LTPs exerted significant antioxidant effects that dramatically reduced damage to hepatocytes by elevating total anti-oxidative capacity (TAOC) in the livers of DEN/PB treated rats. Accordingly, pharmaceutical compositions including an effective amount of one or more modified green tea polyphenols, to reduce, decrease, limit or prevent the symptoms of liver disease in a subject relative to an untreated control subject and a pharmaceutically acceptable excipient are provided. In preferred embodiments the one or more modified green tea polyphenols is in an amount equivalent to about 400 mg/kg body weight of the subject.

A. Green Tea Polyphenols

1. Naturally Occurring Tea Polyphenols

Green tea polyphenols (GTPs) are a naturally-occurring plant product derived from dried tea leaves that may have useful biologically properties. GTPs have shown a great promise in the prevention of human cancers due to their antioxidant activity (Llovet, et al., Lancet, 2003, 362(9399): 1907-1917). This type of compound can potentially be used as natural antioxidant food additive in various products, including dietary oils. GTP is a mixture of biologically active polyphenols mainly comprised of (−)-Epigallocatechin-3-gallate (EGCG), (−)-epigallocatechin (EGC), (−)-epicatechin-3-gallate (ECG), and (−)-epicatechin (EC), in which EGCG is the most abundant constituent (Bishayee, Cancer Treat Rev, 2010, 36(1): 43-53). GTP extract and its biologically active compounds, including EGCG, ECG, EGC and EC, have been shown both in vitro and in vivo to possess antioxidant, anticancer, anti-inflammatory properties.

However, because of the physical and chemical characteristics of GTPs they are hard to be absorbed and used in the grease system. The plasma concentration of GTPs is usually less than 10 μM due to restricted absorption and a high metabolic rate. In animals receiving tea preparations in cancer prevention studies blood levels of EGCG are generally lower than 0.5 μuM, which is much lower than concentrations used in vitro (Bickers, et al., Dermatol., 2000, 27(11): 691-695). Furthermore GTPs are unstable and easy to be oxidized due to the special structure of the tea polyphenols. There have been reports showing excessive amounts of GTP induce organ toxicity (Mihara, et al., Anal. Biochem., 1978, 86(1): 271-278; Eriksson, et al., Environ. Health Perspect, 1983, 49(171-174 ; Jin, et al., Radiology, 2010, 254(1): 129-137).

2. Lipid-Soluble Tea Polyphenols

Due to its limited bioavailability, natural GTP cannot contribute many beneficial effects that have been observed in vitro, including antioxidant, anti-cancer, anti-obesity, anti-atherosclerotic, anti-diabetic, anti-viral, anti-bacterial, anti-fungal effects, as well as neuro-protective activities, when used in vivo (Bickers, et al., J Dermatol, 2000, 27(11): 691-695; Miller, et al., Nutr. Clin. Pract., 2012, 27(5): 599-612; Stagos, et al., Food Chem. Toxicol., 2012, 50(6): 2155-2170; Yang, et al., Cancer Epidemiol. Biomarkers Prev., 1998, 7(4): 351-354; Lee, et al., Cancer Epidemiol. Biomarkers Prev., 2002, 11(10 Pt 1): 1025-1032; Yang, et al., Pharmacol. Res., 2011, 64(2): 113-122). To address the problems of poor absorption and bioavailability, GTP, have been chemically modified by a method using an acylation reaction (such as depicted in Scheme 1), in which the lipid solubility is dramatically increased by esterifying with selected long-chain fatty acids.

Modified GTPs, are known collectively as lipophilic tea polyphenols (LTP) (Formula 1). LTPs have a better lipophilicity, as well as a higher cellular absorption in vivo than GTPs which helps make better use of tea polyphenols' beneficial effects. The lipid-soluble tea polyphenols (LTPs) can be dissolved in oil and many hydrophobic solvents. The biological activity of these LTPs can be stabilized, and their bioavailability increased significantly (Mukhtar, et al., Am J Clin. Nutr., 2000, 71(6 Suppl): 1698S-1702S; discussion 1703S-1694S; Yang, et al., Annu. Rev. Pharmacol. Toxicol., 2002, 42(25-54).

Solubility is a measure of the propensity for a substance (the solute) to dissolve in a liquid to form a homogeneous solution. The “lipid solubility”, as used herein, refers to the saturation concentration in a hydrophobic liquid measured at standard temperature and pressure. The modified green tea polyphenol can have a lipid solubility measured in castor oil that is greater than 1 g/100 ml, for example from 1 g to 100 g per 100 ml, from 5 g to 100 g per 100 ml, or from 5 g to 50 g per 100 ml.

The antioxidant efficacy of LTP may differ from GTP owing to its enhanced cellular absorption in vivo. It has been reported that EGCG-palmitate is 24 times more effective than EGCG when compared for their anti-influenza activities (Kim, et al., Am. J. Physiol. Lung Cell Mol. Physiol., 2003, 285(2): L363-369). It is supposed that LTPs has higher bioavailability by oral administration.

Investigations are currently underway to determine the mechanism of action of lipid soluble tea polyphenols. Although the delivery route of LTPs into the human body is still not clear, it is postulated to be via the chylomicron pathway. If so, LTPs would be associated with lipoprotein particles only, which significantly reduces potential binding with serum proteins, but increases the level in lipoproteins such as LDL prior to internalization by hepatocytes (Halliwell, et al., Br J. Pharmacol., 2004, 142(2): 231-255). Current data demonstrate the greater anti-cancer benefits of LTPs in comparison to GTPs.

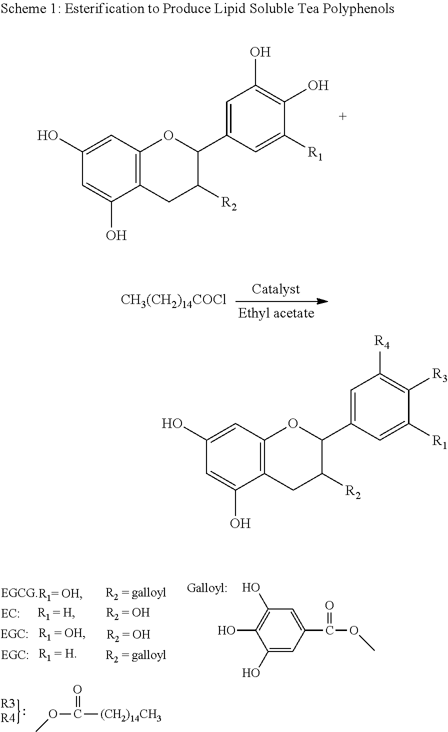

Lipid soluble green tea polyphenols include green tea polyphenols derivatized to increase lipid solubility. For example, the modified green tea polyphenols can include modifications that add one or more aliphatic groups to the green tea polyphenol core. The lipid soluble green tea polyphenol can have the structure in Formula 1 wherein R₁, R₂, R₃, and R₄ are independently hydrogen, hydroxyl, methyl, a halogen atom, or one or more linear, branched, or cyclic alkyl, substituted alkyl, propargyl, alkenyl, substituted alkenyl, alkynyl, substituted alkynyl, carbonyl, substituted carbonyl, carboxyl, substituted carboxyl, amino, substituted amino, amido, substituted amido, sulfonyl, or substituted sulfonyl groups having from 1 to 30 carbon atoms that can be substituted with one or more heteroatoms.

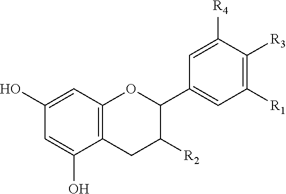

Formula 1: Examples of Lipid Soluble Green Tea Polyphenols

In some embodiments R₁, R₂, R₃, and R₄ are each independently selected from hydrogen, hydroxyl, and linear, branched, or cyclic ether, ketone, or ester groups having from 1 to 30 carbon atoms. The modified green tea polyphenol can have the structure according to Formula 1 wherein R₂ is either hydrogen or a phenyl ester group having from 1 to 30 carbon atoms that can be substituted with one or more heteroatoms and R₁, R₃, and R₄ are each independently either hydrogen, hydroxyl, or one or more linear, cyclic, or branched ester groups having from 1 to 30 carbon atoms. In preferred embodiments the phenyl ester group is galloyl, the structure of which is shown below, or a derivative thereof.

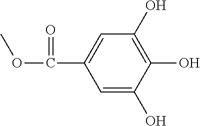

The hydroxyl groups on the galloyl can also be esterified with a fatty acid as described above.

In some embodiments R₁, R₃, and R₄, are independently selected from hydrogen, hydroxyl, and linear ester groups having the structure

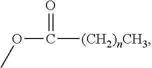

wherein n is an integer from 1 to 28, optionally substituted with one or more heteroatoms. In some embodiments the linear ester group has the structure above wherein n is from 1 to 28, 5 to 25, or 10 to 20. One or more of the bonds in the linear ester group can be unsaturated. In preferred embodiments the linear ester group is a palmitate group (n=14) or a stearate group (n=16). For example, the modified green tea polyphenol can have a structure according to Formula 1 wherein R₂ is hydrogen or galloyl and wherein R₁, R₃, and R₄ are independently hydroxyl, palmitate, or stearate.

B. Antioxidant Enzymes

The generation of reactive oxygen species (ROS) causes continuous oxidative stress in the body and is associated with the pathogenesis of multiple diseases, including cancers (Crawford and Cerutti, 1985). For example, polymorphonuclear neutrophils (PMNs) in an enflamed liver are a major source of ROS and have been associated with liver cancer.

The hydroxyl radical is the most damaging species of ROS and is responsible for base modifications including 5-(hydroxylmethyl) uracil, thymidine glycol and thymine glycol, as well as 8-hydroxydeoxyguanosine (8-OHdG). 8-OHdG is a modified form of guanine responsible for introducing mutations into DNA strands and is used as a marker for oxidative DNA damage.

The major antioxidant enzymes are glutathione peroxidase (GSH-Px), catalase (CAT), superoxide dismutase (SOD) and glutathione S-transferase (GST). These enzymes protect mammalian cells from oxidative stress, for example by reducing hydrogen peroxide and a wide range of organic peroxides and, consequently, reducing the propensity of tissues to develop diseases and malignancy. The combined activity of antioxidant enzymes contributes to the total antioxidant capability (TOAC) in the body.

1. Nuclear Factor-Erythroid 2-Related Factor 2

Nuclear factor-erythroid 2-related factor 2 (Nrf2) is commonly recognized as a redox-sensitive transcription factor that controls the expression of several antioxidant enzymes, protecting cells against oxidative stress from a variety of physiological and environmental stimuli (Katiyar, et al., Int. J. Oncol., 2001, 18(6): 1307-1313). Nrf2 responds to oxidative stress by binding to the antioxidant response element (ARE) in the promoter of genes coding for antioxidant and detoxicating enzymes like NADPH:quinone oxidoreductase 1 and proteins for glutathione synthesis(Sueoka, et al., Ann. NY Acad. Sci., 2001, 928(274-280; Imai, et al., Prev. Med., 1997, 26(6): 769-775). Thus, tea polyphenols can fortify the body's antioxidant defenses by regulation of Nrf2.

The effects of cellular oxidants have been related to activation of transcription factors. The most significant effects of oxidants on signaling pathways have been observed in the nuclear factor erythroid 2-related factor 2 (Nrf2). The mechanisms for activation of Nrf2 have been intensively investigated since its isolation in 1994. A number of endogenous and exogenous stressors have been reported to activate Nrf2 (e.g., ROS). Activation of protein kinases, such as PKC, results in phosphorylation of Nrf2, which enhances the stability and/or release of Nrf2. The activation of Nrf2 results in transcriptional expression of a broad spectrum of protective enzymes including those involved in xenobiotic detoxification, antioxidative response, and proteome maintenance.

2. Peroxiredoxin 6

Peroxiredoxin 6 (P6) is a member of a ubiquitous family of antioxidant enzymes that also control cytokine-induced peroxide levels and thereby mediate signal transduction in mammalian cells. P6 specifically, is involved in redox regulation of the cell, where it may play a role in the regulation of phospholipid turnover as well as in protection against oxidative injury. Recently, the role of P6 in tissue protection against ROS-associated damage was revealed. P6 knockout mice showed impaired wound healing mechanisms after injury or UV-induced damage (Kümin et al., J. Cell Biol., 179:747-760 (2007); Kümin et al., Am. J. Pathol., 169:1194-1205 (2006)).

P6 is one of the ARE-responsive genes regulated by Nrf2 as there is a cis-acting element termed ARE in the promoter of P6 gene, and the ARE within the P6 promoter is a key regulator of basal transcription of the P6 gene (Haqqi, et al., Proc. Natl. Acad. Sci. USA, 1999, 96(8): 4524-4529). P6 acts as a bifunctional enzyme with not only peroxidase function but also phospholipase A₂ activity, which means that P6 has important roles in both antioxidant defense based on its ability to reduce peroxidized membrane phospholipids and in phospholipid homeostasis based on its ability to generate lysophospholipid substrate for the remodeling pathway of phospholipid synthesis (Imai, et al., BMJ, 1995, 310(6981): 693-696). Furthermore, P6 has been shown to be unique compared to its family members due to its ability to reduce phospholipid hyperoxides (Neville, et al., heProstate, (2006), 66(57-69); Xu, et al., Free Radic. Biol. Med., 2012, 52(9): 1543-1551; Shimamoto, et al., Toxicology, 2011, 283(2-3): 109-117). Thus, P6 is considered to be a potential molecular target of chemoprevention and cytoprotection on DEN/PB-induced hepatocarcinogenesis in rats afforded by tea polyphenols.

C. Formulations

The disclosed compositions containing LTPs or a derivative, analog or prodrug, or a pharmacologically active salt thereof can be formulated as pharmaceutical compositions.

Pharmaceutical compositions may be for administration by oral, parenteral (intramuscular, intraperitoneal, intravenous (IV) or subcutaneous injection), transdermal (either passively or using iontophoresis or electroporation), transmucosal (nasal, vaginal, rectal, or sublingual) routes of administration or using bioerodible inserts and can be formulated in unit dosage forms appropriate for each route of administration.

1. Enteral Administration

The compositions can be formulated for oral delivery.

a. Additives for Oral Administration

In a preferred embodiment the LTPs are formulated for oral administration. Oral solid dosage forms are described generally in Remington's Pharmaceutical Sciences, 18th Ed. 1990 (Mack Publishing Co. Easton Pa. 18042) at Chapter 89. Solid dosage forms include tablets, capsules, pills, troches or lozenges, cachets, pellets, powders, or granules or incorporation of the material into particulate preparations of polymeric compounds such as polylactic acid, polyglycolic acid, etc. or into liposomes. Such compositions may influence the physical state, stability, rate of in vivo release, and rate of in vivo clearance of the present proteins and derivatives. See, e.g., Remington's Pharmaceutical Sciences, 18th Ed. (1990), Mack Publishing Co., Easton, Pa. 18042) pages 1435-1712 which are herein incorporated by reference. The compositions may be prepared in liquid form, or may be in dried powder (e.g., lyophilized) form. Liposomal or proteinoid encapsulation may be used to formulate the compositions (as, for example, proteinoid microspheres reported in U.S. Pat. No. 4,925,673). Liposomal encapsulation may be used and the liposomes may be derivatized with various polymers (e.g., U.S. Pat. No. 5,013,556). See also Marshall, K. In: Modern Pharmaceutics Edited by G. S. Banker and C. T. Rhodes, Chapter 10, 1979. In general, the formulation will include the LTP (or chemically modified forms thereof) and inert ingredients which protect the LTP in the stomach environment, and release of the biologically active material in the intestine.

Another embodiment provides liquid dosage forms for oral administration, including pharmaceutically acceptable emulsions, solutions, suspensions, and syrups, which may contain other components including inert diluents; adjuvants such as wetting agents, emulsifying and suspending agents; and sweetening, flavoring, and perfuming agents.

Controlled release oral formulations may be desirable. LTPs, or a derivative, analog or prodrug, or a pharmacologically active salt thereof can be incorporated into an inert matrix which permits release by either diffusion or leaching mechanisms, e.g., gums. Slowly degenerating matrices may also be incorporated into the formulation. Another form of a controlled release is based on the Oros therapeutic system (Alza Corp.), i.e., the drug is enclosed in a semipermeable membrane which allows water to enter and push drug out through a single small opening due to osmotic effects. For oral formulations, the location of release may be the stomach, the small intestine (the duodenum, the jejunum, or the ileum), or the large intestine. Preferably, the release will avoid the deleterious effects of the stomach environment, either by protection of the LTPs (or derivative) or by release of the LTPs (or derivative) beyond the stomach environment, such as in the intestine. To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

b. Chemically Modified Forms for Oral Dosage

LTPs, or a derivative, analog or prodrug, thereof may be chemically modified so that oral delivery of the derivative is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the component or components and increase in circulation time in the body. PEGylation is a preferred chemical modification for pharmaceutical usage. Other moieties that may be used include: propylene glycol, copolymers of ethylene glycol and propylene glycol, carboxymethyl cellulose, dextran, polyvinyl alcohol, polyvinyl pyrrolidone, polyproline, poly-1,3-dioxolane and poly-1,3,6-tioxocane (see, e.g., Abuchowski and Davis (1981) “Soluble Polymer-Enzyme Adducts,” in Enzymes as Drugs. Hocenberg and Roberts, eds. (Wiley-Interscience: New York, N.Y.) pp. 367-383; and Newmark, et al. (1982) J. Appl. Biochem. 4:185-189).

2. Parenteral Administration

In some embodiments, the compositions of LTPs are administered in an aqueous solution, by parenteral injection. The formulation may also be in the form of a suspension or emulsion. In general, pharmaceutical compositions are provided including effective amounts of LTPs, or a derivative, analog or prodrug, or a pharmacologically active salt thereof and optionally include pharmaceutically acceptable diluents, preservatives, solubilizers, emulsifiers, adjuvants and/or carriers. Such compositions include diluents sterile water, buffered saline of various buffer content (e.g., Tris-HCl, acetate, phosphate), pH and ionic strength; and optionally, additives such as detergents and solubilizing agents (e.g., TWEEN®20, TWEEN®80, Polysorbate 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

3. Controlled Delivery Polymeric Matrices

Controlled release polymeric devices can be made for long term release systemically following implantation of a polymeric device (rod, cylinder, film, disk) or injection (microparticles). The matrix can be in the form of microparticles such as microspheres, where peptides are dispersed within a solid polymeric matrix or microcapsules, where the core is of a different material than the polymeric shell, and the peptide is dispersed or suspended in the core, which may be liquid or solid in nature. Unless specifically defined herein, microparticles, microspheres, and microcapsules are used interchangeably. Alternatively, the polymer may be cast as a thin slab or film, ranging from nanometers to four centimeters, a powder produced by grinding or other standard techniques, or even a gel such as a hydrogel.

Either non-biodegradable or biodegradable matrices can be used for delivery of LTPs, although biodegradable matrices are preferred. These may be natural or synthetic polymers, although synthetic polymers are preferred due to the better characterization of degradation and release profiles. The polymer is selected based on the period over which release is desired. In some cases linear release may be most useful, although in others a pulse release or “bulk release” may provide more effective results. The polymer may be in the form of a hydrogel (typically in absorbing up to about 90% by weight of water), and can optionally be cross-linked with multivalent ions or polymers.

The matrices can be formed by solvent evaporation; spray drying, solvent extraction and other methods known to those skilled in the art. Bioerodible microspheres can be prepared using any of the methods developed for making microspheres for drug delivery, for example, as described by Mathiowitz and Langer, J. Controlled Release, 5,13-22 (1987); Mathiowitz, et al., Reactive Polymers, 6, 275-283 (1987); and Mathiowitz, et al., J. Appl. Polymer Sci., 35, 755-774 (1988).

The devices can be formulated for local release to treat the area that is subject to a disease, which will typically deliver a dosage that is much less than the dosage for treatment of an entire body or systemic delivery. These can be implanted or injected subcutaneously, into the muscle, fat, or swallowed.

D. Targeting Moieties

In some embodiments, the composition includes a targeting signal, a protein transduction domain or a combination thereof. The targeting moiety can be attached or linked directly or indirectly to LTPs, or a derivative, analog or prodrug thereof. For example, in preferred embodiments, the targeting moiety is attached or linked to a LTPs delivery vehicle such as a nanoparticle or a microparticle.

The targeting signal or sequence can be specific for a host, tissue, organ, cell, organelle, non-nuclear organelle, or cellular compartment. Moreover, the compositions disclosed here can be targeted to other specific intercellular regions, compartments, or cell types.

In one embodiment, the targeting signal binds to its ligand or receptor which is located on the surface of a target cell such as to bring the LTPs and cell membranes sufficiently close to each other to allow penetration of the LTPs into the cell. Additional embodiments of the present disclosure are directed to specifically delivering LTPs, or a derivative, analog or prodrug, or a pharmacologically active salt thereof to specific tissue or cell types with undesirable oxidative stress. In a preferred embodiment, the targeting molecule is selected from the group consisting of an antibody or antigen binding fragment thereof, an antibody domain, an antigen, a T-cell receptor, a cell surface receptor, a cell surface adhesion molecule, a major histocompatibility locus protein, a viral envelope protein and a peptide selected by phage display that binds specifically to a defined cell.

LTPs can be attached to polymeric particles directly or indirectly through adaptor elements which interact with the polymeric particle. The polymeric particles can microparticles or nanoparticles. Adaptor elements may be attached to polymeric particles in at least two ways. The first is during the preparation of micro- and nanoparticles, for example, by incorporation of stabilizers with functional chemical groups during emulsion preparation of microparticles. For example, adaptor elements, such as fatty acids, hydrophobic or amphiphilic peptides and polypeptides can be inserted into the particles during emulsion preparation. In a second embodiment, adaptor elements may be amphiphilic molecules such as fatty acids or lipids which may be passively adsorbed and adhered to the particle surface, thereby introducing functional end groups for tethering to ligands. Adaptor elements may associate with micro- and nanoparticles through a variety of interactions including, but not limited to, hydrophobic interactions, electrostatic interactions and covalent coupling.

Exemplary targeting signals include a binding moiety such as an antibody or antigen binding fragment thereof specific for a receptor expressed at the surface of a target cell or other specific antigens, such as cancer antigens. Representative receptors include but are not limited to growth factors receptors, such as epidermal growth factor receptor (EGFR; HER1; c-erbB2 (HER2); c-erbB3 (HER3); c-erbB4 (HER4); insulin receptor; insulin-like growth factor receptor 1 (IGF-1R); insulin-like growth factor receptor 2/Mannose-6-phosphate receptor (IGF-II R/M-6-P receptor); insulin receptor related kinase (IRRK); platelet-derived growth factor receptor (PDGFR); colony-stimulating factor-1receptor (CSF-1R) (c-Fms); steel receptor (c-Kit); Flk2/Flt3; fibroblast growth factor receptor 1 (Flg/Cek1); fibroblast growth factor receptor 2 (Bek/Cek3/K-Sam); Fibroblast growth factor receptor 3; Fibroblast growth factor receptor 4; nerve growth factor receptor (NGFR) (TrkA); BDNF receptor (TrkB); NT-3-receptor (TrkC); vascular endothelial growth factor receptor 1 (Flt1); vascular endothelial growth factor receptor 2/Flk1/KDR; hepatocyte growth factor receptor (HGF-R/Met); Eph; Eck; Eek; Cek4/Mek4/HEK; CekS; Elk/Cek6; Cek7; Sek/Cek8; Cek9; Cek10; HEK11; 9 Rorl; Ror2; Ret; Axl; RYK; DDR; and Tie.

In some embodiments, the targeting signal is or includes a protein transduction domain, also known as cell penetrating peptides (CPPS). PTDs are known in the art, and include but are not limited to small regions of proteins that are able to cross a cell membrane in a receptor-independent mechanism (Kabouridis, P., Trends in Biotechnology, (11):498-503 (2003)). The two most commonly employed PTDs are derived from TAT (Frankel and Pabo, Cell, Dec 23;55(6):1189-93 (1988)) protein of HIV and Antennapedia transcription factor from Drosophila, whose PTD is known as Penetratin (Derossi et al., J. Biol. Chem., 269(14):10444-50 (1994)).

### EXAMPLES

### Example 1

### Lipid Soluble Tea Polyphenols Reduce Precancerous Hepatic Lesions

### Materials and Methods

Strategy

Recently, interest in exploring chemoprevention as an approach to the control of cancer has increased. Plant chemicals are gaining much attention in the management of cancer (Yang, et al., Nat Rev Cancer, 2009, 9(6): 429-439). Many naturally occurring agents have been shown to display cancer chemo-preventive potential in a variety of animal models and human disease. The chemo-preventive effect of LTPs has not been investigated against carcinogen-initiated hepatic neoplasia in vivo until very recently. In order to understand the effect of chemo-preventive action of LTPs, a well-described model of HCC was adopted to study the mechanism of anticancer action by evaluating its anti-oxidative damage, anti-proliferative, and anti-fibrosis effects.

Plant Material and Preparation.

LTPs suspensions was purchased from Pulimeidi Chemical Company (11.5%, Production batches: 20091025-1, Hangzhou), using corn oil as solvent. 67.2 ml LTPs was diluted to make the 80 mg/ml work mixture with 32.8 ml corn oil, and then diluted to 8 mg/ml work solution. The liquid was then administered to animals at a dose of 0, 40, and 400 mg/kg body weight in a volume of 0.5 ml/kg body weight.

Chemicals, Kits and Antibodies

N-Nitrosodiethylamine (DEN) was purchased from Sigma-Aldrich (St. Louis, Mo.); gamma-glutamyl transferase (GGT) was purchased from Nanking jiancheng Biological Product Co. (Nanking, china); rabbit anti-GST-P primary antibody and anti-8-hydroxy-2′-deoxuguanosine (8-OHdG) antibody were purchased from Abcam (USA); anti-proliferating cell nuclear antigen (PCNA) antibody from Bio World (USA); 3-diamino-benzidene (DAB) and immunohistochemical kits were purchased from Beijing Golden Bridge Biotechnology Company Ltd. (Beijing, china). masson staining kit was purchased from Loogene Biotechnology Company Ltd. (Beijing, china).

Animals

Male Sprague-Dawley rats (147-157 g) were procured from Zhejiang Academy of Medical Science. The animals were housed in solid bottom polycarbonated cages (five animals/cage) with standard laboratory conditions (temperature 24±0.5° C., relative humidity 55±5%, and a 12 h dark/light cycle). They were acclimatized to the conditions for two weeks before commencement of the experiment.

Statistical Analysis

Data were analysed using SPSS 18.0 and represented as mean±standard error (SE). Multi-group comparisons were evaluated using one-way-analysis of variance (ANOVA) followed by Least-significant difference (LSD) in post-hoc test for the experiment groups. Statistical probability of p<0.05 was considered significant. Masson's trichrome staining data were analyzed by the Kruskal-Wallis tests.

Experimental Design

Rats were divided into four groups and each group consisting of 25 animals. They were subjected to the following treatments: Group 1 (Normal Control): Animals were fed normally throughout the experimental period and injected with single dose of saline (0.9%). Group 2 (Solvent Control): DEN/PB-treated animals, which were given LTPs 0 mg/kg alone by gavage 5 time weekly throughout the experimental period. Group 3 (LTPs 40 mg/kg): DEN/PB-treated animals, which were administrated with LTPs 40 mg/kg by gavage 5 time weekly throughout the experimental period. Group 4 (LTPs 400 mg/kg): DEN/PB-treated animals, which were given LTPs 400 mg/kg by gavage 5 time weekly throughout the experimental period. HCC was induced in groups 2-4 with single intraperitoneal injection of DEN at a dose of 150 mg/kg body weight after two weeks. After given DEN, the promoter PB was incorporated into the drinking water of groups 2-4 at the concentration of 0.05%, which was drunk ad libitum by rats for up to 28 weeks. A schematic representation of the experimental system is given in FIG. 1.

The inhibitory effect of LTPs on the appearance of early hepatic preneoplastic events, employing a two-stage carcinogenic model combining DEN and PB were analyzed. The data demonstrated, for the first time, that LTPs inhibited the incidence of liver carcinogenesis and prevented DBN/PB-induced hepatotoxicity.

Morphology, Morphometry and Histology

After the rats were sacrificed, their livers were promptly excised, weighed and then examined macroscopically on the surface as well as in 3mm cross-sections for gross visible persistent nodules (PNs). Representative sections from right, left and caudate lobes of each liver were taken, then fixed in 10% of neutral formaldehyde, paraffin embeded, sliced, and stained with HE were examined under microscopy. Masson trichrome staining was performed to assess changes in collagen deposition and fibrosis. Scoring was established according to the criteria in Table 1.

Electron Microscopy

The liver tissue was fixed in 2.5% glutaraldehyde buffered for 2 hours, then stored at 4° C. It was fixed in 1% cold osmium tetraoxide for 1 hour and flushed using 0.1M PBS at pH 7.2 for 15min. Ultrathin sections were obtained from specimens embedded in Lowicryl K4M resin after dehydration through graded ethanol series, substitution and polymerization at graded temperature series. Ultrathin sections were obtained using an Ultracut microtome (Leica, Vienna, Austria). Sections were mounted on 400-mesh collodion-carbon-coated nickel grids and examined with a Joel Electron Microscope (JAPAN) operating at 80 kV.

Immunohistochemistry (IHC)

5 μm sections of paraffin-embedded liver tissue were analyzed for expression of PCNA and 8-OHdG by immunohistochemistry. Briefly, sections were deparaffinized in xylene and dehydrated through graded ethanol. After washing with PBS three times, the sections were incubated with 3% hydrogen peroxide for 10 min at room temperature to inhibit endogenous peroxidase activity. After rinsing in PBS three times, the sections were heated with citrate buffer solution (pH 7.2-7.6) for 15 min at 98° C. for antigen retrieval. Subsequently, the sections were then treated with 5% normal goat serum binding for 40 min. Antibody against 8-OHdG, PCNA or GST-P was incubated overnight. The second day sections were treated with the immunohistochemistry kit according to the manufacturer's instructions. Incubation with appropriate secondary antibody was followed by direct diaminobenzidine staining and light counterstaining with hematoxylin.

Quantitation of foci

The number and area of GST-P foci larger than 200 μm in diameter in the liver sections at the early stages of tumor promotion were measured as previously reported (Bishayee, et al., Carcinogenesis, 2011, 32(6): 888-896). The GST-P foci were counted in 5 randomly selected fields under 100× magnification. Then the number and areas of foci /cm² were calculated. The brownish yellow nuclei particles represented the positive signal of PCNA, which were counted in 6 randomly selected fields under 400× magnification. PCNA labeling index (LI) was expressed as the number of PCNA-positive hepatocytes×100/total number of hepatocytes analyzed. The brownish yellow particles represented positive 8-OHdG, which were detected in 6 randomly selected fields under 400× magnification. Relative expression was calculated using image-plus software.

### Results

Body Weight

To investigate the mechanism by which LTPs attenuated hepatocarcinogenesis, the extent of cell proliferation in DEN induced tumorigenesis in the presence or absence of LTPs was examined. Cell proliferation is considered to play a pivotal role in all phases of carcinogenesis with multiple genetic changes. The mean body weight gain of different groups is shown in Table 3.

There was a decrease in the final body weight of LTPs 40, 400 mg/kg groups as compared to the 0 mg/kg group. The average liver weight of LTPs 0 mg/kg group was significantly increased compared to that of normal control group (p<0.05).

Liver and Relative Liver Weight

A similar correlation was found for the liver organ coefficients between these two groups. However, the liver weight and relative liver weight in 400 mg/kg group was found to be significantly decreased than that 0 mg/kg group (p<0.05). There was no statistical difference between 40 mg/kg group and 0 mg/kg group for the liver weights and relative liver weights (Table 3).

Histopathological Analysis

Histopathological analyses of liver sections from various experimental groups of animals are depicted in FIG. 2. The livers of normal control animals (group 1) showed normal hepatocellular architecture mainly consists of normal cytoplasm and small uniform nuclei radially arranged around the central vein (FIG. 2A). Animals subjected to DEN/PB and solvent (Group 2) showed a loss of normal architecture with irregular shaped hepatocytes and increased of nucleoplasm ration and hepatic sinusoid. Moreover, extensive steatosis cells with masses of vacuole in cytoplasm which were clearly distinguishable from the surrounding normal parenchyma was observed (FIG. 2B). Rats treated with LTPs at a dose of 40 mg/kg only marginally improved the hepatocellular architecture which has ameliorative cell degeneration as compared to group 2. (FIG. 2C). In the group that received LTPs at 400 mg/kg (group 4), a moderate improvement in hepatocellular structure was evidenced. Steatosis decreased significantly compared to group 2.The hepatocellular with regular arrangement which size of nuclei was essentially the same as observed in normal cells (FIG. 2D).

Ultrastructural Analysis of the Livers

In electron microscope preparations, the cell surface of the hepatic cells from control group was smooth, with large spherical nucleus and nucleoli showing fibril granular network structure. The cytoplasm showed a granular appearance. There were profuse amount of rough endoplasmic reticulum especially around the nuclear envelope and between the rounded mitochondria. The hepatic sinusoids were thin-walled with discontinuous layer of endothelial and Kupffer cells. The endothelial cells were extremely thin with an electron-lucent cytoplasm. In animals treated with LTPs 0 mg/kg, the nuclei were found to contain a large amount of scattered areas of heterochromatin. The cytoplasm of the hepatic cells contained a fairly large amount of vacuole and fracture of the endoplasmic reticulum rough and smooth endoplasmic reticulum with many damaged mitochondria However, in LTPs 400 mg/kg treated rats, most cells with characteristic large rounded nuclei and large nucleoli contain relatively complete organelles, especially the clear vision of rough endoplasmic reticulum and mitochondria. The cytoplasmic vacuoles were sparse and the fat droplets disappeared in group 4.

Histological findings clearly showed that the normal architecture of hepatic tissue was damaged due to DENA/PB treatment. The hyperplastic nodular hepatocytes formed solid aggregates of mono or multicellular thickness with “hyperbasophilic foci” around the portal vein. The clear and acidophilic cells commonly form altered hepatocyte foci, which represent small preneoplastic focal lesions, leading to malignant transformation in later stages of carcinogenesis with the formation of neoplastic nodules and ultimately HCC (Yang, et al., Arch Toxicol, 2009, 83(1): 11-21). In DEN/PB group, the majority of hepatocyte nodules consisted of a mixture of preneoplastic, neoplastic and diverse intermediate cells. On the other hand, exposure to long term LTPs treatment elicited a reduced hepatocyte aggregation and with a reversal of heterogeneity towards normal cellular architecture.

Effects of LTPs on Fibrosis

Liver fibrosis is the consequence of chronic liver injury from a variety of origins, including exposure to DEN. Liver fibrosis can lead to cirrhosis, liver failure, portal hypertension, and liver cancer (Lambert, et al., Food Chem. Toxicol., 2010, 48(1): 409-416). So the phenomenon of liver fibrosis would occur in the formation of precancerous lesions. The effect of LTPs during the progression of hepatic fibrosis was investigated. Results of the Masson's trichrome assay showed that the normal control (group 1) did not demonstrate histological evidence of steatosis, inflammation or fibrosis, whereas DEN/PB and solvent (Group 2) resulted in liver fibrosis (Table 2). The 400 mg/kg group had mild fibrosis, with fibrosis scoring found to be statistically significant compare to Group 2 (Table 3). The result shows that 400 mg/kg LTPs could reduce the degree of fibrosis significantly.

Effect of LTPs on DEN-Induced foci of Altered Hepatocyte Formation and GST-p Expression.

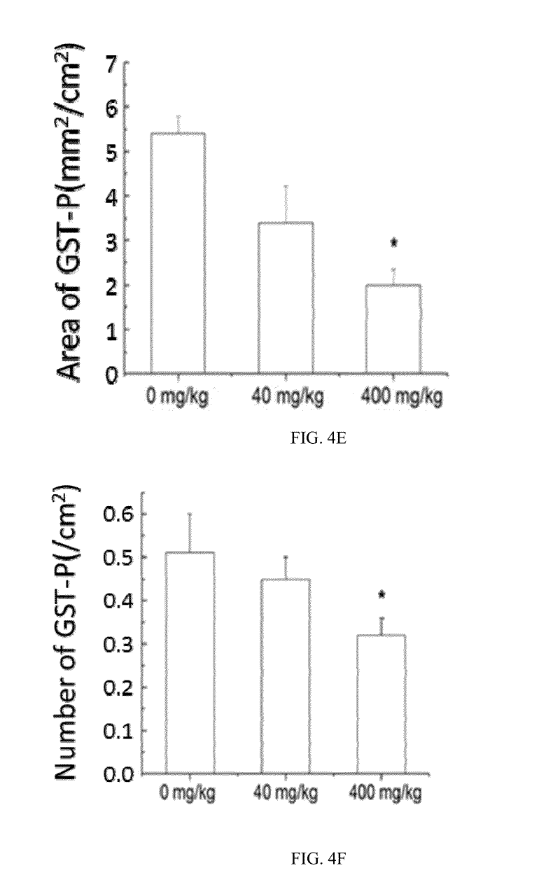

Glutathione S Transferase (GST-P) is specifically expressed during rat hepatocarcinogenesis, and has been used as a reliable tumor marker for experimental hepatocarcinogenesis in rats (29.34). The expression levels of GST-P in the four treatment groups at 30 weeks were therefore examined. As expected, GST-P was not expressed in the normal control liver samples by immunohistochemical (FIG. 4A). The GST-P-positive area and number became more evident in the DEN/PB-treated liver (Group B) (FIG. 4B). Expression levels of GST-P were modified by the treatment with 40 mg/kg (Group C) LTP, but differences were not statistically significant (FIG. 4C). It was observed that the GST-P-positive area and number was significantly reduced by the treatment with 400 mg/kg LTP compared to that group 2 (FIG. 4D). This LTPs mediated reduction of foci of altered hepatocytes formation was closely associated by significant decrease in the number and area of GST-P positive foci, which are reliable and sensitive markers of preneoplasia and neoplasia (Bonkovsky, et al., Ann. Intern. Med., 2006, 144(1): 68-71; Inoue, et al., Cell Stress Chaperones, 2011, 16(6): 653-662.

Effect of LTPs on Proliferation of Hepatic Cells

PCNA is an essential regulator of the cell cycle, whose expression has been a useful tool to study cell proliferation (Na, et al., Food Chem Toxicol, 2008, 46(4): 1271-1278). The PCNA protein has wider physiological functions such as DNA replication, DNA repair and chromatin assembly; maximum expression of PCNA is thought to occur in the S phase (Lee, et al., Cancer Lett., 2005, 224(2): 171-184). Although its expression is increased in proliferating cells, it is also present in non-proliferating cells as most tumors actively undergo DNA repair. The detection of PCNA by immunohistochemical techniques is a common way to study the proliferative activity of transformed cells (Bishayee, et al., Cancer Prev. Res (Phila.), 2010, 3(6): 753-763).

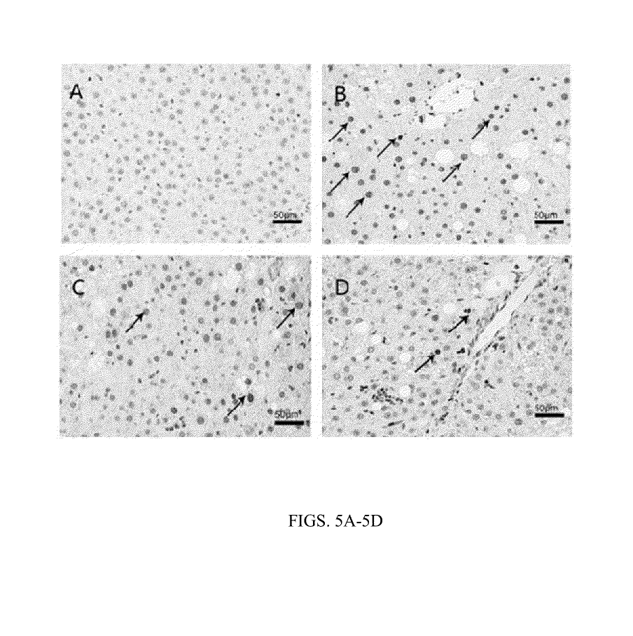

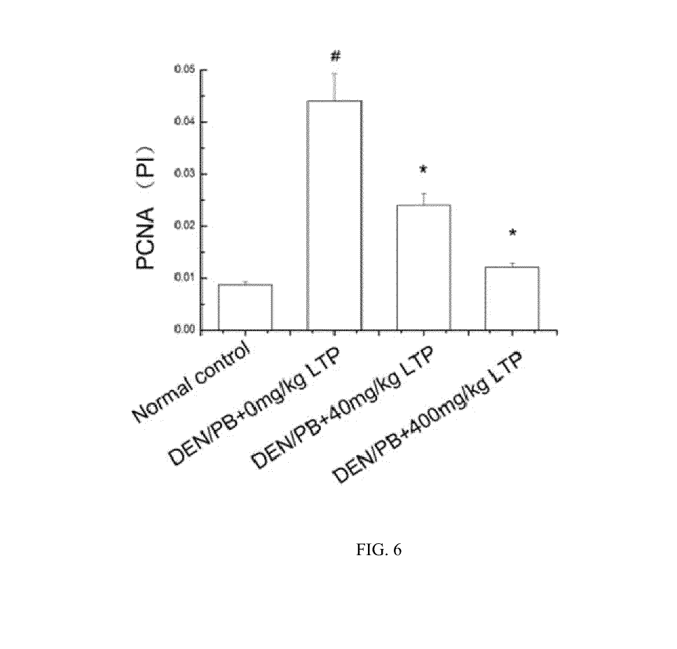

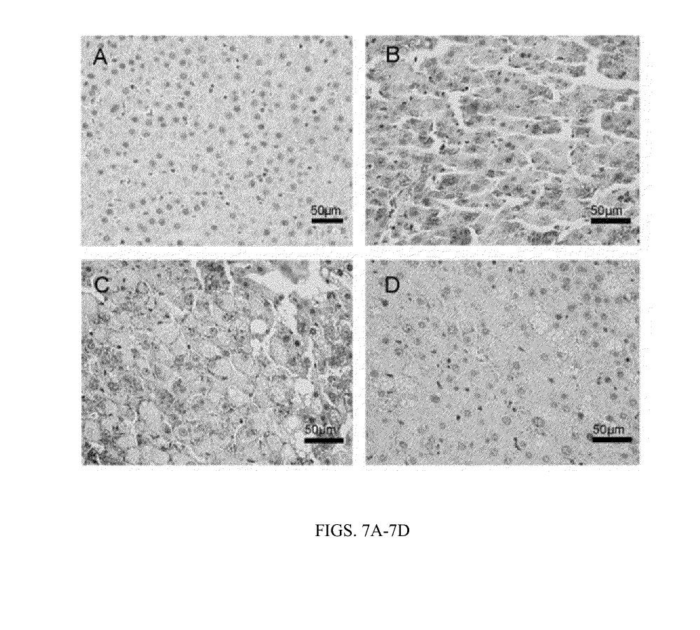

Expression levels of PCNA in the liver among the four treatment groups were compared. As expected, immunohistochemical analysis revealed that PCNA-positive cells were scarcely observed in the normal control liver (FIG. 5A). PCNA-positive cells were significantly increased following treatment with DEN/PB (FIG. 5B) and significantly suppressed after treatment with LTPs 40 (FIG. 5C) or 400 mg/kg (FIG. 5D) compared to group 2 (FIG. 5B). Graphical representation of PCNA levels is shown in FIG. 6.

Among the physiological alterations cancer cells undergo as they continue to grow are an increase in cell proliferation and DNA damage mechanisms. The number of PCNA positive hepatocytes increased in group 2 compared to the normal group are regarded as proliferating cells, especially in S phase. These results suggest that DEN/PB has a proliferating potential to hepatocytes and a tumor promoting potential in the livers of rats. LTP remarkably reduced the number of PCNA positive cells, indicating that it is capable of suppressing malignant proliferation of hepatocytes in experimental hepatocarcinogenesis through its anti-proliferative activity.

Effect of LTPs on Oxidative DNA Damage.

8-hydroxy-2′-deoxyguanosine (8-OHdG) is a well-known oxidative guanine and a marker of oxidative damage in the cellular components (Thoppil, et al., Curr. Cancer Drug Targets, 2012, 12(9): 1244-1257). There are more than 100 types of oxidative base modification in mammalian DNA (Kaspar, et al., Free Radic. Biol. Med, 2009, 47(9): 1304-1309), and 8-hydroxy-2-deoxyguanosine (8-OHdG) is one of the most abundant oxidative DNA damages (Senthil Kumaran, et al., Exp. Gerontol., 2008, 43(3): 176-183). The results of recent immunohistochemical studies indicate 8-OHdG expression in the cell nucleus (Srividhya, et al.,

Int. J. Dev. Neurosci., 2008, 26(2): 217-223; Wang, et al., J. Biol. Chem., 2003, 278(27): 25179-25190). Some researchers observed that the positive expression of mitochondrial DNA damage was in the cytoplasm (Wang, et al., Free Radic. Biol. Med., 2004, 37(11): 1736-1743).

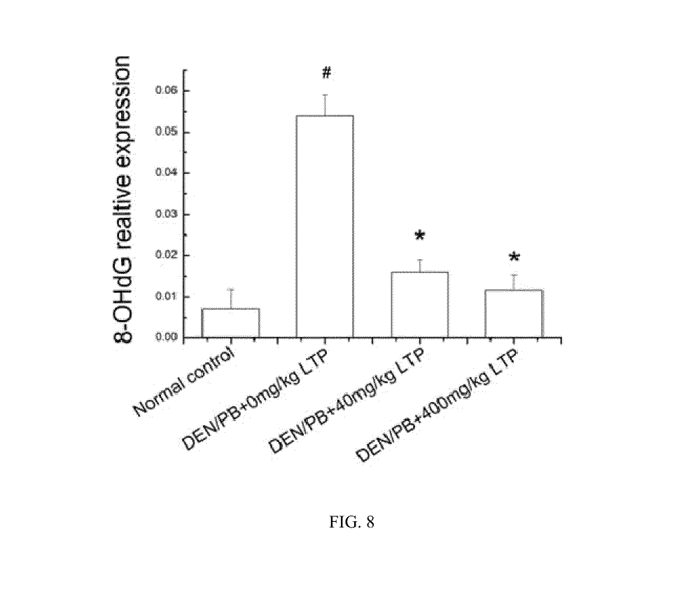

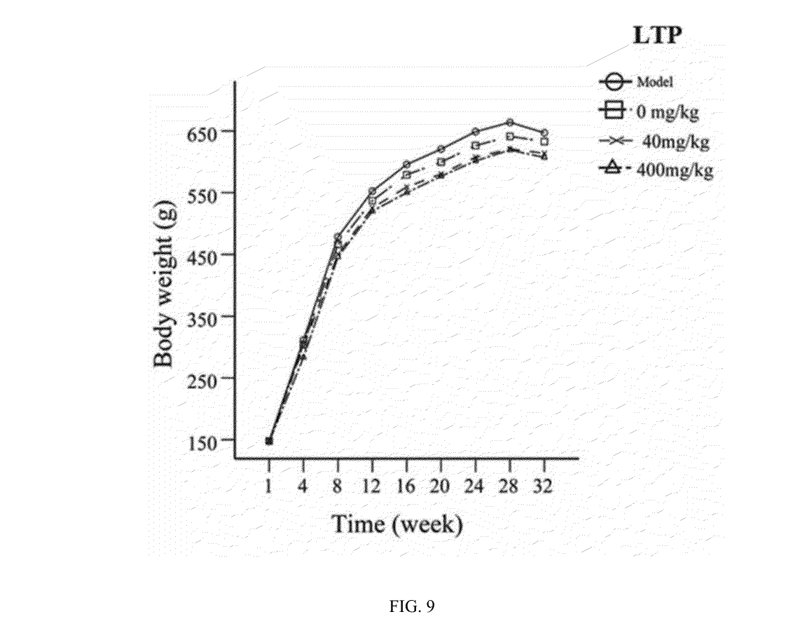

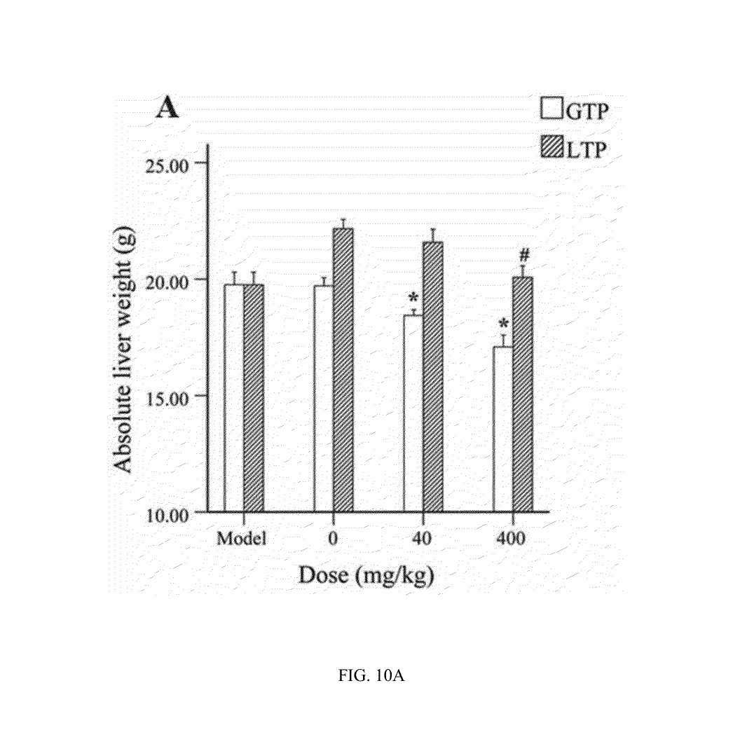

8-hydroxy-2′-deoxyguanosine (8-OHdG) is a marker of oxidative DNA damage. There was no positive immune-labeling of 8-OHdG observed in the normal control group (FIG. 7A). However, under DEN/PB treat, there were many cells with 8-OHdG expression in the cytoplasm. 8-OHdG expression was significantly suppressed after the treatment with LTPs 40 mg/kg (FIGS. 7C), 400 mg/kg (FIGS. 7D), as compared to group 2 (FIG. 7B). Graphical representation of PCNA levels is shown in FIG. 8.

Immunohistochemical positive expression in cell cytoplasm was observed, in combination with electron microscope results indicating that mitochondria damage is serious. Liver cells are rich in mitochondria, and the brown of the cytoplasm could be evidence of mitochondrial DNA oxidative damage.

8-OHdG levels significantly increased in group 2. The data presented here and in recent studies suggested that 8-OHdG production resulting from the ROS generation can result in enhanced induction of preneoplastic lesions in the liver of rats given DEN/PB. Generation of ROS is thought to have a bilateral character; one being to damage the cell component, and the other being to enhance the proliferation of cells (Nagy, et al., Am. J. Physiol. Heart Circ. Physiol., 2006, 291(6): H2636-2640). In this study, PCNA and 8-OHdG increased in the two-stage hepatocarcinogenesis model of rats, and suggested that ROS generation enhanced tumor promotion. The ability of 40 mg/kg or 400 mg/kg LTPs to reduce the number of proliferative cells and DNA damaged cells has been implicated in the chemopreventive action of this polyphenol against DEN/PB-initiated rat hepatocellular carcinogenesis.

### Example 2

### Antioxidant Effects of Lipophilic Tea Polyphenols

Animals and Diet

Pathogen-free male Sprague-awley rats, initially weighing 140-155g, were obtained from the Zhejiang Experimental Animal Center, China and were maintained at a conventional animal facility in Laboratory Animal Center of Zhejiang University. The animals were housed in automatically controlled conditions with a 12 h light-dark cycle, at 23-25° C. and 50-60% relative humidity. All rats were given standard rodent pellet food and water ad libitum. The experimental protocol was approved by the Laboratory Animal Center, and strictly adhered to during the entire study.

Hepatocarcinogenesis Model

The experimental hepatocarcinogenesis was initiated by diethylnitrosamine (DEN) and promoted by phenobarbital (PB). DEN were injected intraperitoneally (i.p.) with a dose of DEN 150mg/kg body weight dissolved in saline once. At the DEN rejection day, the promoter PB was incorporated into the drinking water at the concentration of 0.05%.

Experimental Design and Treatment

Rats were randomly divided into 8 groups with 25 animals in each, consisting of group I (normal group), group II (model group, or DEN/PB-alone group), group III-V (GTP 0, 40, 400 mg/kg groups), group VI-VIII (LTP 0, 40, 400 mg/kg groups). At week 2 Group I was administered normal saline intraperitoneally once, meanwhile the other groups were given DEN/PB to build hepatocarcinogenesis model. Before the administration of the carcinogen, rats were pretreated with GTP and LTP for 2 weeks. GTP groups were given GTP at a dose of 0, 40, 400 mg/kg body weight 5 times weekly by oral gavage for 30 weeks, sterile water was given to the 0 mg/kg group. LTP groups were given LTP at a dose of 0, 40, 400 mg/kg body weight 5 times weekly by oral gavage for the same period, corn oil was given to the 0 mg/kg group.

Weighing and Sample Preparation

Body weight was measured once a week, and food consumption was measured once a month. Necropsy was performed immediately after bleeding the femoral artery to death at the end point after starvation for 16 h. Livers, kidneys, spleens, and lungs were weighed and stored at -80° C. for biochemical analyses. Representative liver slices were taken immediately immersed in 4% paraformaldehyde and stored at 4° C. for histological and immunohistochemical analyses.

Determination of Antioxidant Activity

Liver samples were homogenized for 5min in saline (1:9 w/v) using a homogenizer. After centrifugation at 4000 g for 10min at 4° C., supernatants were used to analyze the levels of MDA, T-AOC and the activity of GSH-Px. The level of MDA, T-AOC and the activity of GSH-Px in liver tissues were measured using kits (all from Nanjing Jiancheng Bioengineering Institute, Nanjing, China) according to the manufacturer's instructions. The T-AOC was a representative of enzyme and non-enzyme antioxidant in the body. These antioxidants reduced the ferric ion (Fe³⁺) to ferrous ion (Fe²⁺). The latter combined with phenanthroline and produced a stable chelate, which could be measured by spectrophotography at 520 nm.

Immunohistochemical Analysis of Nrf2 and Peroxiredoxin 6

After deparaffinization, target retrieval by a hot water bath (95° C.) in citrate buffer and incubation in 0.3% H202 and normal goat serum, sections were subjected to immunohistochemistry with a rabbit polyclonal anti-rat Nrf2 antibody (Sigma-Aldrich, 1:200, 4° C., overnight) and a rabbit polyclonal anti-P6 antibody (Abcam, 1:10000, 4° C., overnight). All specimens were lightly counterstained with hematoxylin. The numbers of Nrf2 and P6 positive cells per 1000 cells were measured with the use of Image Pro Plus.

Statistical Analysis

Unless otherwise specified, all data are presented as Means±Standard error of mean (S.E.) and significance of the differences between mean values was determined by one-way analysis of variance (ANOVA) using a commercial software program SPSS (Statistical Product and Service Solutions, SPSS Inc, Chicago, Ill., USA) except the body data. The body data was analyzed by general linear model/repeated measures. A probability level less than 0.05 was used as a criterion for significance.

Results

General Observations

None of the rats in normal group and model group died during the observation period, whereas seven rats from GTP 400 mg/kg group died without 45183100 GRU2014-007 clear causes. Nine animals from various experimental groups died due to gavage operation mistake. During the entire study period, no differences in food intake were noted among the various experimental groups. No hepatic nodules or tumors were visible in the liver of all groups.

Body Weight

Body weight increased in all experimental groups, from an average of 366.2 g to an average of 499.4 g after 30 weeks. The body weight gain of GTP 400 mg/kg group decreased notably (P<0.05) when compared to the control, while LTP treatments don't show differences within the 3 doses groups (FIG. 9).

Liver and Relative Liver Weight

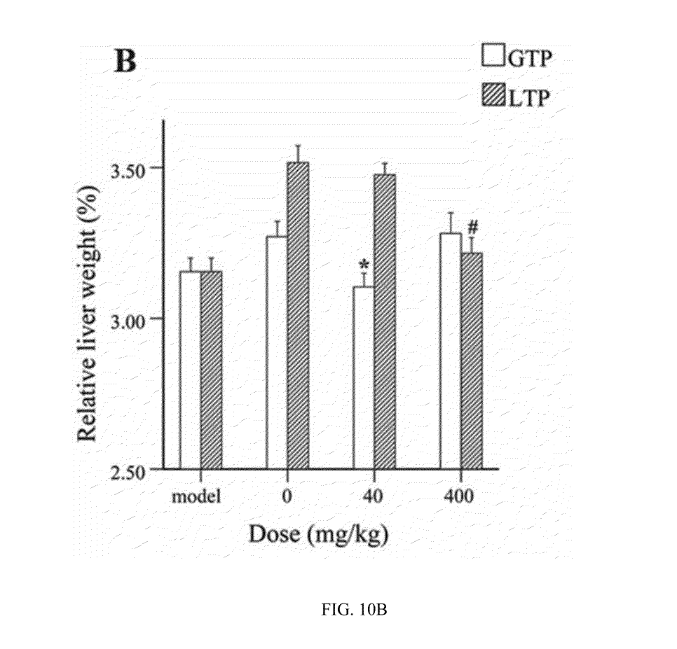

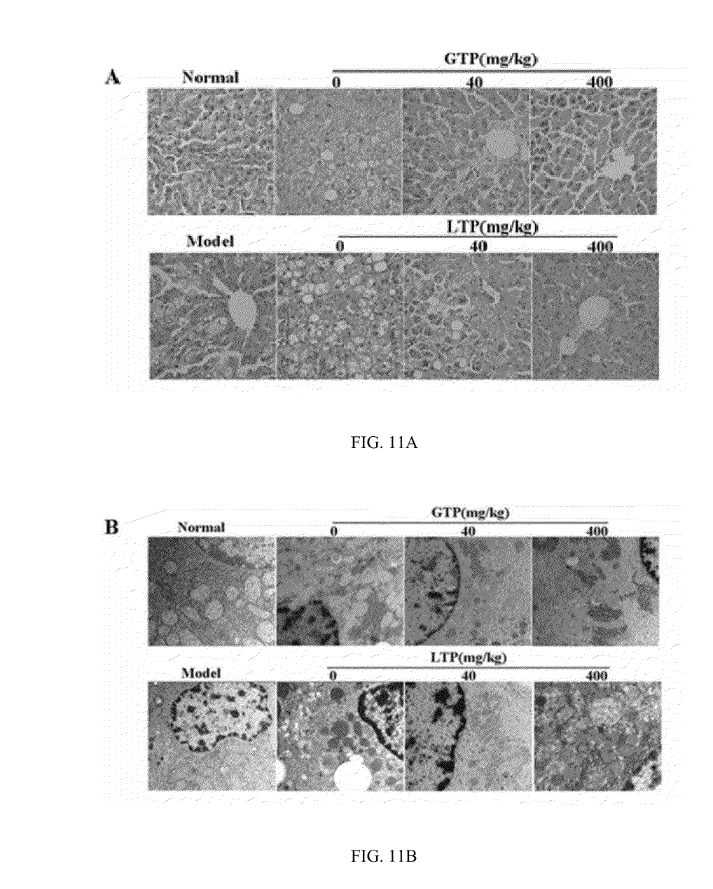

GTP at 40 mg/kg and 400 mg/kg significantly (P<0.05) reduced average liver weight compared with the control group. LTP at 40 mg/kg did not alter liver weight but when at 400 mg/kg significantly (P<0.05) reduced liver weight (FIG. 10A). Significant decreases (P<0.05) in relative liver weight of rats in GTP 40 mg/kg group and LTP 400 mg/kg group were noted (FIG. 10B).

Effects of GTP/LTP on Hepatic Histology

The liver of normal group revealed normal parenchymal cells with granulated cytoplasm and small uniform nuclei radially arranged around the central vein. Animals in only DEN-treated group (model group and 0 mg/kg groups) showed a significant loss of hepatocyte architecture as seen by the presence of extensive vacuolation in the cytoplasm with masses of acidophilic or eosinophilic material. Additionally, the 0 mg/kg LTP group revealed numerous lipid vacuoles. LTP treatments led to gross and noticeable improvement in hepatocellular architecture at the dose of 40 and 400 mg/kg in a dose-response manner (FIG. 11A). The electron microscopy shows exposure to DEN/PB produced an increase of vacuoles in cytoplasm, as well as damages to the mitochondria. The GTP treatment cannot reverse theses damages, while the LTP at a dose of 400 mg/kg can moderately reverse these damages (FIG. 11B). Microphotograph from LTP 400 mg/kg group is the most similar to that of normal group. Histopathological and electron microscopic examination of liver tissue confirmed the protective effect of LTP.

Effects of GTP/LTP on Antioxidant Defense System

LTP at 40 mg/kg and 400 mg/kg significantly increased (P<0.05) total antioxidant capacity (T-AOC) and the same trends were observed in glutathione peroxidase (GSH-Px) activity in liver tissues when compared to the 0 mg/kg group. Whereas GTP only at a high dose of 400 mg/kg can significantly (P<0.05) increase T-AOC and GSH-Px activity (FIG. 12).

Effects of GTP/LTP on Antioxidant Protein Nrf2 and P6 Expression

Immunohistochemical detection of cellular antioxidant protein nuclear factor-erythroid 2-related factor 2 (Nrf2) indicated that Nrf2 positive cells increased significantly (P<0.05) in the livers after exposure to GTP 40 mg/kg, whereas there were few Nrf2 positive cells after exposure to LTP 40 and 400 mg/kg. Many of the immuno-positive cells for Nrf2 were observed in the nucleus, indicating activation of Nrf2 and its subsequent nuclear translocation (FIGS. 13A, C).

Immunohistochemical analysis of cellular antioxidant protein Peroxiredoxin 6 (P6) revealed that very limited expression of P6 in the liver sections of normal animals, while there was a slight increase in the expression of P6 in hepatic cells of DEN-alone and solvent treatments (0 mg/kg group). No significant difference was observed in P6 positive stained cells in the livers of rats from different dose groups of GTP. The P6 positive cells elevated dramatically (P<0.05) only in the livers of LTP 40 mg/kg supplemented rats (FIGS. 13B, D).

I claim:

1. A method of treating liver disease in a subject, comprising
a) administering to the subject an effective amount of one or more lipid soluble green tea polyphenols to reduce, decrease, limit or inhibit one or more symptoms of liver disease relative to an untreated control subject.

2. The method of claim 1 wherein the liver disease is selected from the list consisting of liver cancer, fatty liver and liver cirrhosis.

3. The method of claim 1 wherein the liver disease is liver cancer.

4. The method of claim 1 wherein the liver cancer is selected from the list consisting of hepatocellular carcinoma (HCC), cholangiocarcinoma, hemangioendotheliomas, mesenchymal tissue cancers, sarcoma, hepatoblastoma, angiocarcinoma, hemangiocarsinoma and lymphoma of the liver.

5. The method of claim 4 wherein the liver cancer is hepatocellular carcinoma (HCC).

6. The method of claim 1 wherein one or more symptoms of liver disease are taken from the list consisting of increased abdominal mass, fatigue, abdominal pain, cachexia, jaundice, obstructive syndromes including lymphatic blockage and accumulation of ascites, anemia, back pain and any combination thereof

7. The method of claim 1 further comprising administering to the subject one or more additional pharmaceutically active agents.

8. The method of claim 7 wherein the one or more pharmaceutically active agents is a chemotherapeutic agent.

9. The method of claim 8 wherein the one or more chemotherapeutic agents are taken from the list consisting of sorafenib, erlotinib hydrochloride, cisplatin, cetuximab, sunitinib, bevacizumab, carboplatin, oxaliplatin, mechlorethamine, cyclophosphamide, chlorambucil, vincristine, vinblastine, vinorelbine, vindesine, taxol and derivatives thereof, irinotecan, topotecan, amsacrine, etoposide, etoposide phosphate, teniposide, epipodophyllotoxins, trastuzumab, and rituximab and combinations thereof.

10. The method of claim 1, wherein the subject is asymptomatic.

11. The method of claim 1, wherein the one or more lipid soluble green tea polyphenols are administered at a dose of 400 mg/kg body weight five times weekly.

12. A method of prophylactically treating liver disease in a subject at risk of developing liver disease, comprising
a) selecting a subject with an increased risk of developing liver disease; and
b) administering to the subject an effective amount of one or more lipid soluble green tea polyphenols, to reduce the risk of developing liver disease relative to an untreated control.

13. The method of claim 12 wherein the subject with an increased risk of developing liver disease has one or more of the risk factors taken from the list consisting of inherited metabolic disease, liver cirrhosis, infection with Hepatitis B virus, infection with Hepatitis C virus, alcohol abuse, non-alcoholic fatty liver disease, diabetes mellitus type 2, obesity, hepatocellular adenomas, exposure to afflatoxins, exposure to environmental carcinogens, recreational drug abuse, tobacco smoking or any combination thereof.

14. The method of claim 13 wherein one risk factor is infection with Hepatitis C virus.

15. A pharmaceutical composition comprising
a) an amount of one or more lipid soluble green tea polyphenols equivalent to about 400 mg/kg body weight; and
b) a pharmaceutically acceptable excipient.

16. The pharmaceutical composition of claim 15 further comprising one or more additional pharmaceutical agents.

17. The pharmaceutical composition of claim 16, wherein one or more additional pharmaceutical agents is a chemotherapeutic agent.

18. The pharmaceutical composition of claim 17, wherein one or more chemotherapeutic agents is taken from the list consisting of sorafenib, erlotinib hydrochloride, cisplatin, cetuximab, sunitinib, bevacizumab, carboplatin, oxaliplatin, mechlorethamine, cyclophosphamide, chlorambucil, vincristine, vinblastine, vinorelbine, vindesine, taxol and derivatives thereof, irinotecan, topotecan, amsacrine, etoposide, etoposide phosphate, teniposide, epipodophyllotoxins, trastuzumab, and rituximab and combinations thereof.
